(19) United States
(12) Patent Application Publication
(10) Pub. No.: US 2026/0151367 A1
(43) Pub. Date: Jun. 4, 2026

(54) KIT TO PROMOTE DEVELOPMENTAL MYELINATION

(71) Applicant: SOCIÉTÉ DES PRODUITS NESTLÉ S.A., Vevey (CH)

(72) Inventors: Nora SCHNEIDER, Les Monts-de-Corsier (CH); Sean DEONI, Dighton, MA (US); Fabio MAINARDI, Lausanne (CH)

(21) Appl. No.: 19/103,982

(22) PCT Filed: Aug. 14, 2023
(86) PCT No.: PCT/EP2023/072380

§ 371 Date: Feb. 14, 2025

(30) Foreign Application Priority Data
Aug. 18, 2022 (EP) 22191027.6
Jul. 19, 2023 (EP) 23186445.5

(51) Int. Cl. A61K 31/198; A61K 31/015; A61K 31/136; A61K 31/202; A61K 31/4415; A61K 31/51; A61K 31/525; A61K 31/685; A61K 31/688; A61K 31/702; A61K 31/733

(52) U.S. Cl. CPC A61K 31/198; A61K 31/015; A61K 31/136; A61K 31/202; A61K 31/4415; A61K 31/51; A61K 31/525; A61K 31/685; A61K 31/688; A61K 31/702; A61K 31/733

(57) ABSTRACT

The present invention relates to a kit of nutrient blend compositions for young individuals, in particular children aged between 0 and 66 months, each one of the compositions having age-specific ingredients. The kit of nutrient blend compositions is specifically aimed at promoting myelination of targeted brain regions, in particular the social brain, of children at specific ages and stages of brain development.

### CROSS REFERENCE TO RELATED APPLICATIONS

The present application is a National Stage of International Application No. PCT/EP2023/072380 filed Aug. 14, 2023, which claims priority to European Patent Application No. 22191027.6 filed Aug. 18, 2022, and European Patent Application No. 23186445.5 filed Jul. 19, 2023, the entire contents of which are incorporated herein by reference.

### FIELD OF THE INVENTION

The present invention relates to nutritional compositions for young individuals, in particular children aged between 0 and 66 months. In particular, the invention provides a kit of nutrient blend compositions for children, each one of the compositions having age-specific ingredients. The kit of nutrient blend compositions is specifically aimed at promoting myelination of targeted brain regions, in particular the social brain, of children at specific ages and stages of brain development.

### BACKGROUND OF THE INVENTION

Brain development occurs during the aging of a child and is most essential in early childhood. It places exceptionally high demands on the supply of key nutrients to a child. Failure to meet these nutrient demands during this crucial period may result in sub-optimal neurodevelopment, in particular brain development.

A neurodevelopmental process that plays a key role in brain development, and that has been shown to be impacted by nutritional factors, is myelination, the process by which naked axons are ensheathed in myelin. This process, and in particular the trajectory it follows during brain development, plays a central role in determining brain structure and connectivity, in particular the amount, temporal and/or spatial distribution of myelinated matter throughout the brain.

The relevance of brain structure and connectivity, in particular the amount, temporal and/or spatial distribution of myelinated matter throughout the social brain is well documented. In itself myelin in the brain provides an insulating sheath along neuronal axons permitting much faster conduction of nerve impulses. However, it is brain structure and connectivity, in particular the amount, temporal and/or spatial distribution of myelin throughout the brain, that affects brain connectivity e.g. via what pathway and how quickly and efficiently, messages in the form of neural impulses are communicated within the brain and in particular between different brain regions. This interbrain communication can play a role in particular in socio-emotional skills, performance and development, as they are coordinated in the brain by multiple brain regions, known as social brain, that have to efficiently communicate with each other to perceive, process and act on social, emotional and social-emotional stimuli.

The conditions and diseases related to impaired myelination are well-disclosed in prior art:

| Disease | Phenotype | Reference |
|---|---|---|
| Fetal Alcohol Syndrome | Dysmyelination | Darbinian N, Selzer M E. Oligodendrocyte pathology |
| | | in fetal alcohol spectrum disorders. Neural Regen |
| | | Res. 2022 Mar.; 17(3): 497-502. doi: 10.4103/1673- |
| | | 5374.314294. PMID: 34380877; PMCID: |
| | | PMC8504395. |
| Epileptic Encephalopathy, Early | Abnormal | Yuksel, Z., Yazol, M., Gumus, E. Pathogenic |
| Infantile, 76; EIEE76 | myelination | homozygous variations in ACTL6B cause DECAM |
| | | syndrome: developmental delay, epileptic |
| | | encephalopathy, cerebral atrophy, and abnormal |
| | | myelination. Am. J. Med. Genet. 179A: 1603-1608, |
| | | 2019 |
| Leukodystrophy, | Hypomyelination | Kusk, M. S., Damgaard, B., Risom, L., Hansen, B., |
| Hypomyelinating, 4; HLD4, | | Ostergaard E. Hypomyelinating leukodystrophy due |
| Mitochondrial HSP60 | | to HSPD1 mutations: a new |
| Chaperonopathy | | patient. Neuropediatrics 47: 332-335, 2016. |
| Charcot-Marie-Tooth Disease | Demyelinating | Djordjevic, D., Pinard, M., Gauthier, M.-S., Smith- |
| | and axonal | Hicks, C., Hoffman, T L., Wolf, N I., Oegema, R., van |
| | neuropathy | Binsbergen, E., Baskin, B., Bernard, G., Fribourg, S., |
| | | Coulombe, B., Yoon, G. De novo variants in POLR3B |
| | | cause ataxia, spasticity, and demyelinating |
| | | neuropathy. Am. J. Hum. Genet. 108: 186-193, |
| | | 2021. |
| Autism Spectrum Disorders | Hypomyelination, | Galvez-Contreras A Y, Zarate-Lopez D, Torres-Chavez |
| | Hypermyelination, | A L, Gonzalez-Perez O. Role of Oligodendrocytes and |
| | Delay | Myelin in the Pathophysiology of Autism Spectrum |
| | | Disorder. Brain Sci. 2020 Dec. 8; 10(12): 951. doi: |
| | | 10.3390/brainsci10120951. PMID: 33302549; |
| | | PMCID: PMC7764453. |
| Fragile X Syndrome | Delay | Swanson M R, Wolff J J, Shen M D, et al. Development |
| | | of White Matter Circuitry in Infants With Fragile X |
| | | Syndrome. JAMA Psychiatry. 2018; 75(5): 505-513. |
| | | doi: 10.1001/jamapsychiatry.2018.0180 |
| Down Syndrome | Delay | Ábrahám H, Vincze A, Veszprémi B, Kravják A, |
| | | Gömöri É, Kovács G G, Seress L. Impaired |
| | | myelination of the human hippocampal formation |
| | | in Down syndrome. Int J Dev Neurosci. 2012 |
| | | Apr.; 30(2): 147-58. doi: |
| | | 10.1016/j.ijdevneu.2011.11.005. Epub 2011 Dec. 6. |
| | | PMID: 22155002. |
| 15q11.2 BP1-BP2 | White Matter | Silva A I, Ulfarsson M O, Stefansson H, Gustafsson O, |
| | Changes | Walters G B, Linden D E J, Wilkinson L S, Drakesmith |
| | | M, Owen M J, Hall J, Stefansson K. Reciprocal White |
| | | Matter Changes Associated With Copy Number |
| | | Variation at 15q11.2 BP1-BP2: A Diffusion Tensor |
| | | Imaging Study. Biol Psychiatry. 2019 Apr. |
| | | 1; 85(7): 563-572. doi: |
| | | 10.1016/j.biopsych.2018.11.004. Epub 2018 Nov. |
| | | 19. PMID: 30583851; PMCID: PMC6424871. |
| ADHD | Hypomyelination | Kilpatrick, Lisa & Alger, Jeffry & O'Neill, Joseph & |
| | | Joshi, Shantanu & Narr, Katherine & Levitt, Jennifer |
| | | & O'Connor, Mary. (2022). Impact of prenatal |
| | | alcohol exposure on intracortical myelination and |
| | | deep white matter in children with attention deficit |
| | | hyperactivity disorder. Neuroimage: Reports. 2. |
| | | 100082.10.1016/j.ynirp.2022.100082. |
| Canavan Disease | Demyelination | Lotun A, Gessler D J, Gao G. Canavan Disease as a |
| (Leukodystrophy) | | Model for Gene Therapy-Mediated Myelin Repair. |
| | | Front Cell Neurosci. 2021 Apr. 23; 15: 661928. doi: |
| | | 10.3389/fncel.2021.661928. PMID: 33967698; |
| | | PMCID: PMC8102781. |
| Alexander Disease | Myelin loss | Gómez-Pinedo U, Duran-Moreno M, Sirerol-Piquer |
| | | S, Matias-Guiu J. Myelin changes in Alexander |
| | | disease. Neurologia (Engl Ed). 2018 Oct.; 33(8):526- |
| | | 533. English, Spanish. doi: |
| | | 10.1016/j.nrl.2017.01.019. Epub 2017 Mar. 22. |
| | | PMID: 28342553. |
| Krabbe Disease (KRB) Globoid Cell | | Husain, A. M., Altuwaijri, M., Aldosari, M. Krabbe |
| Leukodystrophy | | disease: neurophysiologic studies and MRI |
| | | correlations. Neurology 63: 617-620, 2004. |
| Leukodystrophy: | Demyelination | Wolf, N. I., ffrench-Constant, C. & van der Knaap, |
| Metachromatic | | M. S. Hypomyelinating leukodystrophies - |
| leukodystrophy | | unravelling myelin biology. Nat Rev Neurol 17, 88- |
| Adrenoleukodystrophy | | 103 (2021). https://doi.org/10.1038/s41582-020- |
| Krabbe disease | | 00432-1 |
| Pelizaeus-Merzbacher | | |
| disease | | |
| Refsum disease | | |
| Vanishing white matter | | |
| Leukoencephalopathy with | | |
| brainstem and spinal cord | | |
| involvement and elevated lactate | | |
| levels | | |
| Biotin-responsive basal | | |
| ganglia disease | | |
| Wilson disease | | |
| Fabry disease | | |
| Alexander disease | | Gómez-Pinedo U, Duran-Moreno M, Sirerol-Piquer |
| Radiation | | S, Matias-Guiu J. Myelin changes in Alexander |
| Chemotherapy | | disease. Neurologia (Engl Ed). 2018 Oct.; 33(8): 526- |
| (methotrexate, cyclosporine, | | 533. English, Spanish. doi: |
| cytosine-arabinoside) | | 10.1016/j.nrl.2017.01.019. Epub 2017 Mar. 22. |
| Extrapontine myelinolysis | | PMID: 28342553. |
| Neoplastic: | | |
| Lymphoma | | |
| Astrocytoma | | |
| Medulloblastoma | | |
| Metastases | | |
| Langerhans cell histiocytosis | | |
| Genetic/metabolic: | | |
| Inborn errors of metabolism | | |
| Amino acid and organic | | |
| aciduria | | |
| GM2 gangliosidosis | | |
| Mitochondrial: | | |
| Myoclonic epilepsy with | | |
| ragged red fibers (MERRF) | | |
| Mitochondrial | | |
| encephalomyopathy with lactic | | |
| acidosis and stroke-like episodes | | |
| (MELAS) | | |
| Leber hereditary optic | | |
| neuropathy (LHON) | | |
| Leigh syndrome | | |
| Kearns-Sayre syndrome | | |
| DNA polymerase gamma | | |
| (POLG)-related disorders | | |
| Nutritional: | | |
| Vitamin B12, vitamin E, or | | |
| folate deficiency | | |
| Celiac disease | | |
| Wernike-Korsakoff | | |
| Acute disseminated | | |
| encephalomyelitis (ADEM) 10 | | |
| Acute transverse myelitis (ATM) | | |
| 10 Optic neuritis 11 Neuromyelitis | | |
| optica (NMO) 11 Acquired | | |
| demyelinating syndrome (ADS) | | |

While nutrient recommendations are typically targeted at overall health and development, very limited longitudinal data exists for brain benefits during development.

The impact of some nutrients and their compositions on promoting overall brain myelination of children are known. Nutritional composition and infant formula promoting myelination of the overall brain is described in patent documents WO2017102710, WO2017102712, WO2017102714, WO2017102715, WO2017102717, WO2017102718, WO2017102719, WO2017102720, WO2017108500, WO2017167415, WO2017167416, WO2017167417, WO2017167419, WO2017167420, WO2017167895, WO2017167896, WO2017167897, WO2017167898, WO2017167899.

However, recent studies (Schneider et al., Child Development: 2022: 93: 359-371) revealed that changes in myelination, specifically amount of myelin, in specific brain regions, in particular in the social brain, during brain development at different ages is associated with socio-emotional development.

Accordingly, there is a need to identify nutrients necessary to promote age-dependent brain development, more specifically myelination, of targeted brain regions, in particular of social brain.

Further there is a need to provide age- and brain stage appropriate nutritional compositions having age-specific ingredients for use to promote myelination of a targeted brain region of children at different developmental stages of a child, in particular of social brain.

The aim of the present invention is to provide optimal nutrient blend compositions having age-specific ingredients to promote myelination of targeted brain regions, in particular social brain, of young individuals, in particular children at specific ages.

### SUMMARY OF THE INVENTION

The object is solved by means of the independent claims.

The dependent claims further develop the central idea of the invention.

The inventors found that at different age stages of a child's development, the brain regions associated with socio-emotional development are sensitive for intake of different age- and stage-specific nutrients that promote myelination of these regions and, consequently, that those specific brain regions are not sensitive to other nutrients.

Thus, in a first aspect the present invention provides an age-tailored kit of nutrient blend compositions to be sequentially administrated to a young individual for use to promote myelination of targeted brain regions comprising at least two of the following compositions:
a) a nutrient blend composition A to be administrated to a young individual at age from 0 to 20 months comprising at least three ingredients chosen from the group consisting of vitamin B1, vitamin B2, vitamin B6, zinc, iron, copper, histidine, isoleucine, lysine, leucine, vitamin A, folate, magnesium, phosphorus, potassium, selenium, 3′-SL, alanine, cystine, glutamic acid, phenylalanine, proline, serine, threonine, tyrosine, valine;
b) a nutrient blend composition B to be administrated to a young individual at age from 20 to 30 months comprising at least three ingredients chosen from the group consisting of DPA, ARA, sphingomyelin, phosphatidylcholine, octadecatetraenoic acid, oligofructose, 6′-SL, stearidonic acid, beta cryptoxanthin, alpha lactalbumin, gangliosides, phosphatidylinositol, 3′-SL, eicosatetraenoic acid;
c) a nutrient blend composition C to be administrated to a young individual at age from 30 to 66 months comprising at least three ingredients chosen from the group consisting of DHA, sphingomyelin, phosphatidylcholine, histidine, isoleucine, leucine, valine, alpha carotene, folic acid, alpha lactalbumin, gangliosides, phosphatidylinositol, phosphatidylethanolamine, eicosenoic acid, stearidonic acid, DPA, 3′-SL, 6′-SL, alanine, arginine, aspartic acid, cystine, glutamic acid, glycine, lysine, methionine, phenylalanine, proline, serine, threonine, tryptophan, tyrosine, alpha tocopherol, octadecatetraenoic acid, eicosapentaenoic acid, phosphorus, phosphatidylserine.

The present invention further provides an age-tailored kit of nutrient blend compositions to be sequentially administrated to a young individual for use to promote myelination of targeted brain regions wherein the targeted brain region is at least one region of the social brain.

In a further aspect, the present invention relates to the kit as described above wherein the young individual is a human, a pet, an infant, a young child or a child.

The present invention further provides a method for promoting brain myelination of targeted brain regions of a young individual comprising sequential administering to a young individual the kit as described above.

In a further aspect, the present invention relates to a staged feeding regimen of a young individual comprising the steps of
a) administering the composition A as described above at age from 0 to 20 months,
b) administering the composition B as described above at age from 20 to 30 months,
c) administering the composition C as described above at age from 30 to 66 months.

In a further aspect, the present invention relates to a use of the staged feeding regimen as described above to promote myelination of targeted brain regions.

In a further aspect, the present invention relates to a use of the staged feeding regimen as described above to promote myelination of targeted brain regions.

In another aspect, the present invention relates to the use of the staged feeding regimen as described above wherein the targeted brain region is at least one region of the social brain.

### BRIEF DESCRIPTION OF FIGURES

FIG. 1 shows associations for untargeted nutrients and brain myelination.

FIG. 2 shows random Forest prediction on test set for untargeted nutrients across ages.

### DETAILED DESCRIPTION OF THE INVENTION

For a complete understanding of the present invention and the advantages thereof, reference is made to the following detailed description of the invention.

### Definitions

As used herein, the following terms have the following meanings.

Within the context of the present invention, the term “nutrient” or “nutrients” is intended to comprise both macronutrients (for example carbohydrates, proteins or fats) and micronutrients (for example minerals or vitamins) and components thereof (e.g. fatty acids, amino acids) for the human body.

Within the context of the present invention, the expression “nutritional composition(s)” refers to composition(s) which nourishes a subject. This nutritional composition is usually to be taken enterally, orally, parenterally or intravenously. Preferably, a nutritional composition is for oral use.

Moreover, “nutritional composition(s)” may refer to liquids, powders, gels, pastes, solids, concentrates, suspensions, or ready-to-use forms of enteral formulas, oral formulas, formulas for infants, formulas for pediatric subjects, formulas for children, growing-up milks and/or formulas for adults, porridges and/or infant cereals, baby food, pet food.

The term “sequentially” means that the compositions of the kit are administered in a successive manner such that composition A of the kit is administered at a first age and/or brain stage period without compositions B and/or C, and composition B is administered at a second age period and/or brain stage (subsequent to the first age period) without compositions A and/or C, and composition C is administered at a third age period and/or brain stage (subsequent to the first and second age periods) without compositions A and/or B.

The term “social brain” refers to brain regions associated with socio-emotional skills, performance or development, such as understanding and interacting with others, experiencing, managing, and expressing a full range of positive and negative emotions, developing close, satisfying relationships with others, including emotional skills, social skills, sociability, social aspects of executive functions, happiness, positive mood, social cognition, social communication, social decision-making, self-regulation, compliance, adaptive functioning, affect, social communication, interaction, autonomy.

The social brain regions may be chosen from any of superior temporal sulcus (STS) involved in recognition of others; anterior cingulate cortex (ACC) involved in emotion and response selectivity; medial prefrontal cortex (mPFC); inferior frontal gyrus (IFG) involved in self-mapping; temporo-parietal junction (TPJ) involved in knowledge of self and others and thinking about mental states; anterior insula, amygdala, both specialized emotion areas; posterior cingulate cortex, insula, medial orbital frontal cortex, middle frontal cortex, rostral middle frontal cortex, precuneus, and superior temporal cortices.

The term “young individual” includes but is not limited to murines, simians, humans, animals and pets.

In human “young individual” refers to children aged between 0 to 66 months.

The term “pet” means any animal which could benefit from or enjoy the compositions provided by the present disclosure. For example, the pet can be an avian, bovine, canine, equine, feline, hircine, lupine, murine, ovine, or porcine animal, but the pet can be any suitable animal.

The term “infant” means a child under the age of 12 months. The term infant includes both infants born at term or who were born preterm.

The expression “young child” means a child aged between one and three years, also called toddler.

The expression “child” means a child aged between one and six years, including toddlers and pre-school children.

Within the context of the present invention, the term “promote” and/or “promoting” indicates a factor or a number of factors causing a certain process to occur.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

Within the context of the present invention, the terms “ingredient” or “ingredients” indicate an edible substance or mixture of substances which comprise or is essentially consisting of a nutrient for the human body.

As used herein, “about” is understood to refer to numbers in a range of numerals, for example the range of −10% to +10% of the referenced number, within −5% to +5% of the referenced number, or in one aspect, within −1% to +1% of the referenced number, and in a specific aspect, within −0.1% to +0.1% of the referenced number. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

The term “liquid” as used here refers to any liquid wherein the powder of a composition can be dispersed into a beverage (e.g. water, fruit juice, milk, etc.) to provide a palatable nutrient liquid for oral administration.

The terms “in particular” or “more particularly” as used herein should not be considered limiting but should be interpreted as being synonymous with “for example” or “especially”.

The term “oligofructose” as used herein refers to fructose oligomers.

“Alpha lactalbumin” refers to a high-quality, easy-to-digest whey protein that comprises 20-25% of total human breast milk (HBM) protein and is the primary protein found in HBM. The structure of alpha lactalbumin is comprised of 123 amino acids and 4 disulfide bridges and the protein has a molecular weight of 14.2K Daltons. Alpha lactalbumin is ideal for lower protein infant formulas due to its high content of essential amino acids, particularly tryptophan. Alpha lactalbumin also represents a source of sphingomyelin according to the present invention.

The term “ganglioside” as used herein indicates an oligoglycosylceramide lipid molecule comprising the residue of a ceramide. When the term ganglioside is used in the present specifications, it may identify a single ganglioside species as well as a mixture of single ganglioside species comprising the residue of ceramide.

The term “sphingomyelin” indicates a lipid molecule wherein the sphingosine backbone is acylated with a fatty acid residue at the amino group (—NH2) and wherein the hydroxyl group at position 1 of the sphingosine backbone is linked to a phospho-choline or phospho-ethanolamine group. When the term sphingomyelin is used in the present specifications, it may identify a single sphingomyelin species as well as a mixture of single sphingomyelin species wherein preferably the fatty acid residue is residue of a C14 to C44 fatty acid non limiting examples of which include Non limiting examples of C14 to C44 saturated or unsaturated fatty acids from which the fatty acid residue may stem include; C14:0, C15:0, C16:0, C18:0, C20:0, C21:0, C22:0, C23:0, C24:1, C25:0, C28:1, C30:2, C30:1, C30:0, C32:3, C32:2, C32:1, C32:0, C33:1, C34:3, C34:2, C34:1, C34:0, C35:2, C35:0, C36:4, C36:3, C36:2, C36:1, C36:0, C37:1, C37:0, C38:4, C38:3, C38:1, C38:0, C39:1, C39:0, C40:2, C40:1, C40:0, C41:2, C41:1, C41:0, C42:47, C42:3, C42:2, C42:1, C42:0, C44:3, C44:1. More Preferably the fatty acid residue is a residue of a C16, C18, C20, C22 or C24 saturated fatty acid.

Within the context of the present invention the term “DHA” identifies docosahexaenoic acid. Within the context of the present invention, the term “DHA” should be intended to identify all the DHA present in the nutritional compositions on the invention, either in free form (as a fatty acid or a physiologically acceptable slat thereof) or comprised in a fatty acid derivative structure.

Within the context of the present invention, the term “ARA” identifies arachidonic acid. Within the context of the present invention, the term “ARA” should be intended to identify all the ARA present in the nutritional compositions on the invention, either in free form (as a fatty acid or a physiologically acceptable salt thereof) or comprised in a fatty acid derivative structure.

Within the context of the present invention, the term “DPA” identifies docosapentaenoic acid.

Within the context of the present invention, the term “Phosphatidylinositole” indicates a compound of formula:

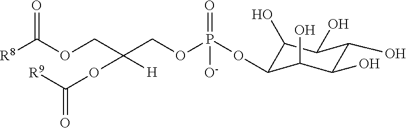

Wherein R8 is a C2 to C43 branched or unbranched acyclic alkyl, or acyclic alkenyl group and, R9 is a C2 to C43 branched or unbranched acyclic alkyl, or acyclic alkenyl group.

More particularly R8 and R9 are, independently of each other, C13 to C43 branched or unbranched acyclic alkyl, or acyclic alkenyl groups which together with their adjacent carbonyl group are correspond to C14 to C44 saturated or unsaturated fatty acid residues, and even more particularly R8 and R9 are, independently of each other, C13 to C23 branched or unbranched acyclic alkyl, or acyclic alkenyl groups which together with their adjacent carbonyl group correspond to C14 to C24 saturated or unsaturated fatty acid residues.

More particularly, R8 and R9 are C13 to C23 branched or unbranched acyclic alkyl, or acyclic alkenyl groups which together with their adjacent carbonyl group are C14 to C24 saturated or unsaturated fatty acid residues, wherein the fatty acids from which the fatty acid residues stem are selected from the group consisting of; C14:0, C15:0, C16:0, C18:0, C20:0, C20:3, C20:4, C21:0, C22:0, C23:0, C24:0, C18:ln-9, C18:2n-6, and C24:ln-9. Even more particularly C18:0, C18:ln-9, C18:2, C20:3, and C20:4.

Within the context of the present invention, the term “Phosphatidylethanolamine” indicates a compound of formula:

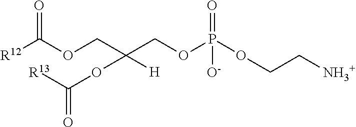

Wherein R12 is a C2 to C43 branched or unbranched acyclic alkyl, or acyclic alkenyl group and, R13 is a C2 to C43 branched or unbranched acyclic alkyl, or acyclic alkenyl group.

More particularly, R12 and R13 are, independently of each other, C13 to C43 branched or unbranched acyclic alkyl, or acyclic alkenyl groups which together with their adjacent carbonyl group correspond to C14 to C44 saturated or unsaturated fatty acid residues, and even more particularly R12 and R13 are, independently of each other, C13 to C23 branched or unbranched acyclic alkyl, or acyclic alkenyl groups which together with their adjacent carbonyl group correspond to C14 to C24 saturated or unsaturated fatty acid residues.

Within the context of the present invention, the term “Phosphatidylcholine” identifies a compound of formula:

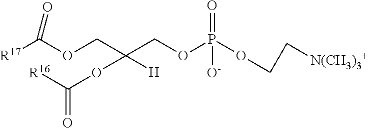

Wherein R16 is a C2 to C43 branched or unbranched acyclic alkyl, or acyclic alkenyl group and, R17 is a C2 to C43 branched or unbranched acyclic alkyl, or acyclic alkenyl group.

More particularly, R16 and R17 are, independently of each other, C13 to C43 branched or unbranched acyclic alky, or acyclic alkenyl groups which together with their adjacent carbonyl group correspond to C14 to C44 saturated or unsaturated fatty acid residues, and even more particularly R16 and R17 are, independently of each other, C13 to C23 branched or unbranched acyclic alkyl, or acyclic alkenyl groups which together with their adjacent carbonyl group correspond to C14 to C24 saturated or unsaturated fatty acid residues.

More particularly, R16 and R17 are C13 to C23 branched or unbranched acyclic alkyl, or acyclic alkenyl groups which together with their adjacent carbonyl group are C14 to C24 saturated or unsaturated fatty acid residues, wherein the fatty acids from which the fatty acid residues stem are selected from the group consisting of; C14:0, C15:0, C16:0, C16:1, C18:0, C20:0, C20:1, C20:3, C20:4, C21:0, C22:0, C22:6, C23:0, C24:0, C18:ln-9, C18:2n-6, and C24:ln-9.

Even more particularly C14:0, C16:0, C18:0, C18:ln-9, C18:2n-6, C20:1, C20:3, C20:4, and C22:6.

Within the context of the present invention, the term “Phosphatidylserine” indicates a compound of formula:

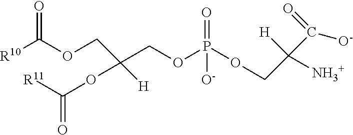

Wherein R10 is a C2 to C43 branched or unbranched acyclic alkyl, or acyclic alkenyl group and, R11 is a C2 to C43 branched or unbranched acyclic alkyl, or acyclic alkenyl group.

More particularly, R10 and R11 are, independently of each other, C13 to C43 branched or unbranched acyclic alkyl, or acyclic alkenyl groups which together with their adjacent carbonyl group correspond to C14 to C44 saturated or unsaturated fatty acid residues, and even more particularly R10 and R11 are, independently of each other, C13 to C23 branched or unbranched acyclic alkyl, or acyclic alkenyl groups which together with their adjacent carbonyl group correspond to C14 to C24 saturated or unsaturated fatty acid residues.

More particularly, R10 and R11 are C13 to C23 branched or unbranched acyclic alkyl, or acyclic alkenyl groups which together with their adjacent carbonyl group are C14 to C24 saturated or unsaturated fatty acid residues, wherein the fatty acids from which the fatty acid residues stem are selected from the group consisting of; C14:0, C15:0, C16:0, C18:0, C20:0, C20:3, C20:4, C21:0, C22:0, C23:0, C24:0, C18:ln-9, C18:2n-6, and C24:ln-9. Even more particularly C18:0, C18:ln-9, C20:4, and C22:6.

Within the context of the present invention the term “3′SL” identifies 3′-sialyllactose.

Within the context of the present invention the term “6′SL” identifies 6′-sialyllactose.

Both 3′SL and 6′SL are human milk oligosaccharides that are useful for promoting neurodevelopment, in particular myelination.

Unless defined otherwise, all technical and scientific terms used herein have the same meaning as commonly understood by one of ordinary skill in the art.

The invention will now be described in further details. It is noted that the various aspects, features, examples and embodiments described in the present application may be compatible and/or combined together.

### Kit of Nutrient Blend Compositions

The inventors examined the structural changes in children's social brains related to myelination and the relation to changes in social emotional development and identified age-/brain stage-appropriate nutrient blend compositions in socio-emotional development that is one of the primary domains of healthy child development. Children develop socio-emotional skills with increasing complexity from birth onwards with developmental tasks and milestones in key domains such as social competence, attachment, emotional competence, self-perceived competence and temperament/personality (Schneider et al., Child Development: 2022: 93: 359-371). Socio-emotional (SE) development during the early years broadly describes the emerging capacity of a child to experience, control, and express feelings while forming close as well as secure interpersonal connections, all in the setting of family, society and cultural anticipations (Metwally et al., PloS one: 2016; 11(7), e0158086).

The inventors found that at different age stages of a child's development, the brain regions associated with socio-emotional development are sensitive for intake of different age- and stage-specific nutrients that promote myelination of these regions and, consequently, that those specific brain regions are not sensitive to other nutrients. Thus, the administration of some of the nutrients known from prior art to promote overall brain myelination during a “non-sensitive” stage may have no effect or even cause negative effect to promoting myelination in targeted brain regions, in particular the social brain regions associated with socio-emotional development, which has not been considered in prior art.

The inventors surprisingly identified three myelin-nutrient windows in the children age range of 1 to 5 years: window 1 from 0-20 months, in particular from 6 to 20 months, window 2 from 20-30 months and window 3 from 30 to 66 months, in particular from 30 to 60 months. These myelin-nutrient windows are aligned with reported myelin and connectivity dynamics that change in the first five years from fast and steep increase of myelination (window 1) to continued but slower increase in myelination (window 3), with in-between window 2 representing the inflection period.

The inventors identified targeted and age-appropriate nutrients for each of the mentioned critical stages of brain development. The sequential administration of nutrient blends, each of these blends tailored to be delivered at a specific stage of the brain development, has the advantage of delivering the nutrients that will efficiently promote myelination of target specific brain regions, and in particular social brain regions associated with socio-emotional development, in alignment with the dynamic needs of the developing brain.

Thus, the invention in a first embodiment relates to an age-tailored kit of nutrient blend compositions to be sequentially administrated to a young individual for use to promote myelination of targeted brain regions comprising at least two of the following compositions:
a) a nutrient blend composition A to be administrated to a young individual at age from 0 to 20 months comprising at least three ingredients chosen from the group consisting of vitamin B1, vitamin B2, vitamin B6, zinc, iron, copper, histidine, isoleucine, lysine, leucine, vitamin A, folate, magnesium, phosphorus, potassium, selenium, 3′-SL, alanine, cystine, glutamic acid, phenylalanine, proline, serine, threonine, tyrosine, valine;
b) a nutrient blend composition B to be administrated to a young individual at age from 20 to 30 months comprising at least three ingredients chosen from the group consisting of DPA, ARA, sphingomyelin, phosphatidylcholine, octadecatetraenoic acid, oligofructose, 6′-SL, stearidonic acid, beta cryptoxanthin, alpha lactalbumin, gangliosides, phosphatidylinositol, 3′-SL, eicosatetraenoic acid;
c) a nutrient blend composition C to be administrated to a young individual at age from 30 to 66 months comprising at least three ingredients chosen from the group consisting of DHA, sphingomyelin, phosphatidylcholine, histidine, isoleucine, leucine, valine, alpha carotene, folic acid, alpha lactalbumin, gangliosides, phosphatidylinositol, phosphatidylethanolamine, eicosenoic acid, stearidonic acid, DPA, 3′-SL, 6′-SL, alanine, arginine, aspartic acid, cystine, glutamic acid, glycine, lysine, methionine, phenylalanine, proline, serine, threonine, tryptophan, tyrosine, alpha tocopherol, octadecatetraenoic acid, eicosapentaenoic acid, phosphorus, phosphatidylserine.

In an embodiment the present invention relates to an age-tailored kit of nutrient blend compositions to be sequentially administrated to a young individual for use to promote myelination of targeted brain regions comprising at least two of the following compositions:
a) a nutrient blend composition A to be administrated to a young individual at age from 0 to 20 months comprising at least three ingredients chosen from the group consisting of vitamin B1, vitamin B2, vitamin B6, zinc, iron, copper, histidine, isoleucine, lysine, leucine, vitamin A, folate, magnesium, phosphorus, potassium, selenium, 3′-SL, alanine, cystine, glutamic acid, phenylalanine, proline, serine, threonine, tyrosine, valine;
b) a nutrient blend composition B to be administrated to a young individual at age from 20 to 30 months comprising at least three ingredients chosen from the group consisting of DPA, ARA, sphingomyelin, phosphatidylcholine, octadecatetraenoic acid, oligofructose, 6′-SL, stearidonic acid, beta cryptoxanthin, alpha lactalbumin, gangliosides, phosphatidylinositol, 3′-SL;
c) a nutrient blend composition C to be administrated to a young individual at age from 30 to 66 months comprising at least three ingredients chosen from the group consisting of DHA, sphingomyelin, phosphatidylcholine, histidine, isoleucine, leucine, valine, alpha carotene, folic acid, alpha lactalbumin, gangliosides, phosphatidylinositol, phosphatidylethanolamine, eicosenoic acid, stearidonic acid, DPA, 3′-SL, 6′-SL, alanine, arginine, aspartic acid, cystine, glutamic acid, glycine, lysine, methionine, phenylalanine, proline, serine, threonine, tryptophan, tyrosine.

In one embodiment of the invention, the nutrient blend composition A is administered to a young individual at age from 6 to 20 months. In another embodiment, the nutrient blend composition C is administered to a young individual at age from 30 to 60 months.

In another embodiment the invention relates to the kit as described above wherein:
a) the nutrient blend composition A is administrated to a young individual at age from 6 to 20 months;
b) the nutrient blend composition B is administrated to a young individual at age from 20 to 30 months;
c) the nutrient blend composition C is administrated to a young individual at age from 30 to 60 months.

In another embodiment the invention relates to the kit as described above wherein:
a) the nutrient blend composition A is administered to a young individual in the stage of the brain development in which there is a fast and steep increase of myelination (window 1);
b) the nutrient blend composition B is administered to a young individual in the stage of brain development representing the inflection period of myelination (window 2);
c) the nutrient blend composition C is administered to a young individual in the stage of the brain development in which there is a continued but slower increase in myelination (window 3).

In one embodiment the invention relates to the kit as described above wherein:
a) said composition A comprises at least three ingredients chosen from the group consisting of vitamin B1, vitamin B2, vitamin B6, zinc, iron, copper, histidine, isoleucine, lysine, leucine.
b) said composition B comprises at least three ingredients chosen from the group consisting of DPA, ARA, sphingomyelin, phosphatidylcholine, octadecatetraenoic acid, oligofructose, 6′-SL.
c) said composition C comprises at least three ingredients chosen from the group consisting of DHA, sphingomyelin, phosphatidylcholine, histidine, isoleucine, leucine, valine.

In one embodiment, the present invention relates to the kit as described above wherein
a) said composition A comprises at least three ingredients chosen from the group consisting of:
   vitamin B1 in a daily amount of at least about 0.8 mg;
   vitamin B2 in a daily amount of at least about 1 mg;
   vitamin B6 in a daily amount of at least about 0.9 mg;
   zinc in a daily amount of at least about 6 mg;
   iron in a daily amount of at least about 8 mg;
   copper in a daily amount of at least about 0.6 mg;
   histidine in a daily amount of at least about 0.2 g;
   isoleucine in a daily amount of at least about 0.3 g;
   lysine in a daily amount of at least about 0.4 g;
   leucine in a daily amount of at least 0.6 g;
   vitamin A in a daily amount of at least about 400 μg;
   folate in a daily amount of at least about 168 μg;
   magnesium in a daily amount of at least about 143 mg;
   phosphorus in a daily amount of at least about 688 mg;
   potassium in a daily amount of at least about 1267 mg;
   selenium in a daily amount of at least about 51 μg;
   3′-SL in a daily amount of at least about 4 mg;
   alanine in a daily amount of at least about 0.3 g;
   cystine in a daily amount of at least about 0.07 g;
   glutamic acid in a daily amount of at least about 2 g;
   phenylalanine in a daily amount of at least about 0.4 g;
   proline in a daily amount of at least about 0.6 g;
   serine in a daily amount of at least about 0.4 g;
   threonine in a daily amount of at least about 0.3 g;
   tyrosine in a daily amount of at least about 0.3 g; and
   valine in a daily amount of at least about 0.5 g;
b) said composition B comprises at least three ingredients chosen from the group consisting of:
   DPA in a daily amount of at least about 3 mg;
   ARA in a daily amount of at least about 0.06 g;
   sphingomyelin in a daily amount of at least about 3 mg;
   phosphatidylcholine in a daily amount of at least about 0.3 g;
   octadecatetraenoic acid in a daily amount of at least about 3 mg;
   oligofructose in a daily amount of at least about 1 g;
   6′-SL in a daily amount of at least about 3 mg;
   stearidonic acid in a daily amount of at least about 3 mg;
   beta cryptoxanthin in a daily amount of at least about 93 mg;
   alpha lactalbumin in a daily amount of at least about 0.3 mg;
   gangliosides in a daily amount of at least about 2 mg;
   phosphatidylinositol in a daily amount of at least about 4 mg; and
   3′-SL in a daily amount of at least about 16 mg;
   eicosatetraenoic acid in a daily amount of at least about 0.1 g;
c) said composition C comprises at least three ingredients chosen from the group consisting of:
   DHA in a daily amount of at least about 0.01 g;
   sphingomyelin in a daily amount of at least about 5 mg;
   phosphatidylcholine in a daily amount of at least about 0.4 g;
   histidine in a daily amount of at least about 0.3 g;
   isoleucine in a daily amount of at least about 0.5 g;
   leucine in a daily amount of at least about 1 g;
   valine in a daily amount of at least about 0.7 g;
   alpha carotene in a daily amount of at least about 214 mg;
   folate in daily amount of at least about 251 μg;
   alpha lactalbumin in a daily amount of at least about 0.1 mg;
   gangliosides in a daily amount of at least about 2 mg;
   phosphatidylinositol in a daily amount of at least about 0.01 g;
   phosphatidylethnolamine in a daily amount of at least about 0.05 g;
   eicosenoic acid in a daily amount of at least about 0.1 g;
   stearidonic acid in daily amount of at least about 3 mg;
   DPA in a daily amount of at least about 8 mg;
   3′-SL in a daily amount of at least about 6 mg;
   6′SL in a daily amount of at least about 2 mg;
   alanine in a daily amount of at least about 0.5 g;
   arginine in a daily amount of at least about 0.5 g;
   aspartic acid in a daily of at least about 1 g;
   cystine in a daily amount of at least about 0.1 g;
   glutamic acid in a daily of at least about 2 g;
   glycine in a daily amount of at least about 0.3 g;
   lysine in a daily amount of at least about 0.7 g;
   methionine in a daily amount of at least about 0.2 g;
   phenylalanine in a daily amount of at least about 0.6 g;
   proline in a daily amount of at least about 1 g;
   serine in a daily amount of at least about 0.6 g;
   threonine in a daily amount of at least about 0.5 g;
   tryptophan in a daily amount of at least about 0.1 g;
   tyrosine in a daily amount of at least about 0.5 g;
   alpha tocopherol in a daily amount of at least about 6 mg;
   octadecatetraenoic acid in a daily amount of at least about 6.8 mg;
   eicosapentaenoic acid in a daily amount of at least about 8 mg;
   phosphorus in a daily amount of at least about 894 mg;
   phosphatidylserine in a daily amount of at least about 18 mg.

In an embodiment, the present invention relates to the kit as described above wherein
a) said composition A comprises at least three ingredients chosen from the group consisting of:
   vitamin B1 in a daily amount of at least about 0.8 mg;
   vitamin B2 in a daily amount of at least about 1 mg;
   vitamin B6 in a daily amount of at least about 0.9 mg;
   zinc in a daily amount of at least about 6 mg;
   iron in a daily amount of at least about 8 mg;
   copper in a daily amount of at least about 0.6 mg;
   histidine in a daily amount of at least about 0.2 g;
   isoleucine in a daily amount of at least about 0.3 g;
   lysine in a daily amount of at least about 0.4 g;
   leucine in a daily amount of at least 0.6 g;
   vitamin A in a daily amount of at least about 400 μg;
   folate in a daily amount of at least about 168 μg;
   magnesium in a daily amount of at least about 143 mg;
   phosphorus in a daily amount of at least about 731 mg;
   potassium in a daily amount of at least about 1267 mg;
   selenium in a daily amount of at least about 51 mg;
   3′-SL in a daily amount of at least about 4 mg;
   alanine in a daily amount of at least about 0.3 g;
   cystine in a daily amount of at least about 0.07 g;
   glutamic acid in a daily amount of at least about 2 g;
   phenylalanine in a daily amount of at least about 0.4 g;
   proline in a daily amount of at least about 0.6 g;
   serine in a daily amount of at least about 0.4 g;
   threonine in a daily amount of at least about 0.3 g;
   tyrosine in a daily amount of at least about 0.3 g; and
   valine in a daily amount of at least about 0.5 g;
b) said composition B comprises at least three ingredients chosen from the group consisting of:
   DPA in a daily amount of at least about 3 mg;
   ARA in a daily amount of at least about 0.06 g;
   sphingomyelin in a daily amount of at least about 3 mg;
   phosphatidylcholine in a daily amount of at least about 0.3 g;
   octadecatetraenoic acid in a daily amount of at least about 3 mg;
   oligofructose in a daily amount of at least about 1 g;
   6′-SL in a daily amount of at least about 3 mg;
   stearidonic acid in a daily amount of at least about 3 mg;
   beta cryptoxanthin in a daily amount of at least about 93 mg;
   alpha lactalbumin in a daily amount of at least about 0.3 mg;
   gangliosides in a daily amount of at least about 2 mg;
   phosphatidylinositol in a daily amount of at least about 4 mg; and
   3′-SL in a daily amount of at least about 16 mg;
c) said composition C comprises at least three ingredients chosen from the group consisting of:
   DHA in a daily amount of at least about 0.01 g;
   sphingomyelin in a daily amount of at least about 5 mg;
   phosphatidylcholine in a daily amount of at least about 0.4 g;
   histidine in a daily amount of at least about 0.3 g;
   isoleucine in a daily amount of at least about 0.5 g;
   leucine in a daily amount of at least about 1 g;
   valine in a daily amount of at least about 0.7 g;
   alpha carotene in a daily amount of at least about 214 mg;
   folate in daily amount of at least about 251 μg;
   alpha lactalbumin in a daily amount of at least about 0.1 mg;
   gangliosides in a daily amount of at least about 2 mg;
   phosphatidylinositol in a daily amount of at least about 0.01 g;
   phosphatidylethnolamine in a daily amount of at least about 0.05 g;
   eicosenoic acid in a daily amount of at least about 0.1 g;
   stearidonic acid in daily amount of at least about 3 mg;
   DPA in a daily amount of at least about 8 mg;
   3′-SL in a daily amount of at least about 6 mg;
   6′SL in a daily amount of at least about 2 mg;
   alanine in a daily amount of at least about 0.5 g;
   arginine in a daily amount of at least about 0.5 g;
   aspartic acid in a daily of at least about 1 g;
   cystine in a daily amount of at least about 0.1 g;
   glutamic acid in a daily of at least about 2 g;
   glycine in a daily amount of at least about 0.3 g;
   lysine in a daily amount of at least about 0.7 g;
   methionine in a daily amount of at least about 0.2 g;
   phenylalanine in a daily amount of at least about 0.6 g;
   proline in a daily amount of at least about 1 g;
   serine in a daily amount of at least about 0.6 g;
   threonine in a daily amount of at least about 0.5 g;
   tryptophan in a daily amount of at least about 0.1 g; and
   tyrosine in a daily amount of at least about 0.5 g.

In an embodiment, the present invention relates to the kit as described above wherein
a) said composition A comprises at least three ingredients chosen from the group consisting of:
   vitamin B1 in a daily amount ranging from about 0.8 to about 2 mg;
   vitamin B2 in a daily amount ranging from about 1 to about 3 mg;
   vitamin B6 in a daily amount ranging from about 0.9 to about 5 mg;
   zinc in a daily amount ranging from about 6 to about 13 mg;
   iron in a daily amount ranging from about 8 to about 29 mg;
   copper in a daily amount ranging from about 0.6 to about 1.1 mg;
   histidine in a daily amount ranging from about 0.2 to about 1 g;
   isoleucine in a daily amount ranging from about 0.3 to about 2 g;
   lysine in a daily amount ranging from about 0.4 to about 3 g;
   leucine in a daily amount ranging from about 0.6 to about 4 g;
   vitamin A in a daily amount ranging from about 400 to about 2600 μg;
   folate in a daily amount ranging from about 168 to about 460 μg;
   magnesium in a daily amount ranging from about 143 to about 369 mg;
   phosphorus in a daily amount ranging from about 688 to about 1687 mg;
   potassium in a daily amount ranging from about 1267 to about 4085 mg;
   selenium in a daily amount ranging from about 51 to about 150 μg;
   3′-SL in a daily amount ranging from about 4 to about 68 mg;
   alanine in a daily amount ranging from about 0.3 to about 2 g;
   cystine in a daily amount ranging from about 0.07 to about 0.4 g;
   glutamic acid in a daily amount ranging from about 2 to about 10 g;
   phenylalanine in a daily amount ranging from about 0.4 to about 2 g;
   proline in a daily amount ranging from about 0.6 to about 3 g;
   serine in a daily amount ranging from about 0.4 to about 2 g;
   threonine in a daily amount ranging from about 0.3 g to about 2 g;
   tyrosine in a daily amount ranging from about 0.3 to about 2 g; and
   valine in a daily amount ranging from about 0.5 to about 2 g;
b) said composition B comprises at least three ingredients chosen from the group consisting of:
   DPA in a daily amount ranging from about 3 mg to about 53 mg;
   ARA in a daily amount ranging from about 0.06 to about 0.4 g;
   sphingomyelin in a daily amount ranging from about 3 to about 55 mg;
   phosphatidylcholine in a daily amount ranging from about 0.3 to about 5 g;
   octadecatetraenoic acid in a daily amount ranging from about 3 to about 33 mg;
   oligofructose in a daily amount ranging from about 1 to about 7 g;
   6′-SL in a daily amount ranging from about 3 to about 23 mg;
   stearidonic acid in a daily amount ranging from about 3 to about 41 mg;
   beta cryptoxanthin in a daily amount ranging from about 93 to about 484 mg;
   alpha lactalbumin in a daily amount ranging from about 0.3 to about 1 mg;
   gangliosides in a daily amount ranging from about 2 to about 15 mg;
   phosphatidylinositol in a daily amount ranging from about 4 to about 59 mg; and
   3′-SL in a daily amount ranging from about 16 to about 113 mg;
   eicosatetraenoic acid in a daily amount ranging from about 0.1 to about 0.3 g;
c) said composition C comprises at least three ingredients chosen from the group consisting of:
   DHA in a daily amount ranging from about 0.01 to about 0.3 g;
   sphingomyelin in a daily amount ranging from about 5 to about 96 mg;
   phosphatidylcholine in a daily amount ranging from about 0.4 to about 4 g;
   histidine in a daily amount ranging from about 0.3 to about 1 g;
   isoleucine in a daily amount ranging from about 0.5 to about 2 g;
   leucine in a daily amount ranging from about 1 to about 4 g;
   valine in a daily amount ranging from about 0.7 to about 3 g;
   alpha carotene in a daily amount ranging from about 214 to about 2087 mg;
   folate in daily amount ranging from about 251 to about 400 μg;
   alpha lactalbumin in a daily amount ranging from about 0.1 to about 1 mg;
   gangliosides in a daily amount ranging from about 2 to about 16 mg;
   phosphatidylinositol in a daily amount ranging from about 0.01 to about 0.2 g;
   phosphatidylethnolamine in a daily amount ranging from about 0.05 to about 0.9 g;
   eicosenoic acid in a daily amount ranging from about 0.1 to about 0.5 g;
   stearidonic acid in daily amount ranging from about 3 to about 70 mg;
   DPA in a daily amount ranging from about 8 to about 46 mg;
   3′-SL in a daily amount ranging from 6 to about 102 mg;
   6′SL in a daily amount ranging from about 2 to about 21 mg;
   alanine in a daily amount ranging from about 0.5 to about 2 g;
   arginine in a daily amount ranging from about 0.5 to about 2 g;
   aspartic acid in a daily amount ranging from about 1 to about 4 g;
   cystine in a daily amount ranging from about 0.1 to about 0.6 g;
   glutamic acid in a daily amount ranging from about 2 to about 11 g;
   glycine in a daily amount ranging from about 0.3 to about 2 g;
   lysine in a daily amount ranging from about 0.7 to about 3 g;
   methionine in a daily amount ranging from about 0.2 to about 1 g;
   phenylalanine in a daily amount ranging from about 0.6 to about 2 g;
   proline in a daily amount ranging from about 1 to about 4 g;
   serine in a daily amount ranging from about 0.6 to about 2 g;
   threonine in a daily amount ranging from about 0.5 to about 2 g;
   tryptophan in a daily amount ranging from about 0.1 to about 0.8 g;
   tyrosine in a daily amount ranging from about 0.5 to about 2 g;
   alpha tocopherol in a daily amount ranging from about 6 to about 18 mg;
   octadecatetraenoic acid in a daily amount ranging from about 6.8 to about 56 mg;
   eicosapentaenoic acid in a daily amount ranging from about 8 to about 127 mg;
   phosphorus in a daily amount ranging from about 894 to about 1475 mg;
   phosphatidylserine in a daily amount ranging from about 18 to about 294 mg.

In an embodiment, the present invention relates to the kit as described above wherein
a) said composition A comprises at least three ingredients chosen from the group consisting of:
   vitamin B1 in a daily amount ranging from about 0.8 to about 2 mg;
   vitamin B2 in a daily amount ranging from about 1 to about 3 mg;
   vitamin B6 in a daily amount ranging from about 0.9 to about 5 mg;
   zinc in a daily amount ranging from about 6 to about 7.5 mg;
   iron in a daily amount ranging from about 8 to about 29 mg;
   copper in a daily amount ranging from about 0.6 to about 1 mg;
   histidine in a daily amount ranging from about 0.2 to about 1 g;
   isoleucine in a daily amount ranging from about 0.3 to about 2 g;
   lysine in a daily amount ranging from about 0.4 to about 3 g;
   leucine in a daily amount ranging from about 0.6 to about 4 g;
   vitamin A in a daily amount ranging from about 400 to about 900 μg;
   folate in a daily amount ranging from about 168 to about 300 μg;
   magnesium in a daily amount ranging from about 143 to about 369 mg;
   phosphorus in a daily amount ranging from about 731 to about 1687 mg;
   potassium in a daily amount ranging from about 1267 to about 4085 mg;
   selenium in a daily amount ranging from about 51 to about 90 mg;
   3′-SL in a daily amount ranging from about 4 to about 68 mg;
   alanine in a daily amount ranging from about 0.3 to about 2 g;
   cystine in a daily amount ranging from about 0.07 to about 0.4 g;
   glutamic acid in a daily amount ranging from about 2 to about 10 g;
   phenylalanine in a daily amount ranging from about 0.4 to about 2 g;
   proline in a daily amount ranging from about 0.6 to about 3 g;
   serine in a daily amount ranging from about 0.4 to about 2 g;
   threonine in a daily amount ranging from about 0.3 g to about 2 g;
   tyrosine in a daily amount ranging from about 0.3 to about 2 g; and
   valine in a daily amount ranging from about 0.5 to about 2 g;
b) said composition B comprises at least three ingredients chosen from the group consisting of:
   DPA in a daily amount ranging from about 3 mg to about 53 mg;
   ARA in a daily amount ranging from about 0.06 to about 0.4 g;
   sphingomyelin in a daily amount ranging from about 3 to about 55 mg;
   phosphatidylcholine in a daily amount ranging from about 0.3 to about 5 g;
   octadecatetraenoic acid in a daily amount ranging from about 3 to about 33 mg;
   oligofructose in a daily amount ranging from about 1 to about 7 g;
   6′-SL in a daily amount ranging from about 3 to about 23 mg;
   stearidonic acid in a daily amount ranging from about 3 to about 41 mg;
   beta cryptoxanthin in a daily amount ranging from about 93 to about 484 mg;
   alpha lactalbumin in a daily amount ranging from about 0.3 to about 1 mg;
   gangliosides in a daily amount ranging from about 2 to about 15 mg;
   phosphatidylinositol in a daily amount ranging from about 4 to about 59 mg; and
   3′-SL in a daily amount ranging from about 16 to about 113 mg;
c) said composition C comprises at least three ingredients chosen from the group consisting of:
   DHA in a daily amount ranging from about 0.01 to about 0.3 g;
   sphingomyelin in a daily amount ranging from about 5 to about 96 mg;
   phosphatidylcholine in a daily amount ranging from about 0.4 to about 4 g;
   histidine in a daily amount ranging from about 0.3 to about 1 g;
   isoleucine in a daily amount ranging from about 0.5 to about 2 g;
   leucine in a daily amount ranging from about 1 to about 4 g;
   valine in a daily amount ranging from about 0.7 to about 3 g;
   alpha carotene in a daily amount ranging from about 214 to about 2087 mg;
   folate in daily amount ranging from about 251 to about 400 μg;
   alpha lactalbumin in a daily amount ranging from about 0.1 to about 1 mg;
   gangliosides in a daily amount ranging from about 2 to about 16 mg;
   phosphatidylinositol in a daily amount ranging from about 0.01 to about 0.2 g;
   phosphatidylethanolamine in a daily amount ranging from about 0.05 to about 0.9 g;
   eicosenoic acid in a daily amount ranging from about 0.1 to about 0.36 g;
   stearidonic acid in daily amount ranging from about 3 to about 70 mg;
   DPA in a daily amount ranging from about 8 to about 46 mg;
   3′-SL in a daily amount ranging from 6 to about 102 mg;
   6′SL in a daily amount ranging from about 2 to about 21 mg;
   alanine in a daily amount ranging from about 0.5 to about 2 g;
   arginine in a daily amount ranging from about 0.5 to about 2 g;
   aspartic acid in a daily amount ranging from about 1 to about 4 g;
   cystine in a daily amount ranging from about 0.1 to about 0.6 g;
   glutamic acid in a daily amount ranging from about 2 to about 11 g;
   glycine in a daily amount ranging from about 0.3 to about 2 g;
   lysine in a daily amount ranging from about 0.7 to about 3 g;
   methionine in a daily amount ranging from about 0.2 to about 1 g;
   phenylalanine in a daily amount ranging from about 0.6 to about 2 g;
   proline in a daily amount ranging from about 1 to about 4 g;
   serine in a daily amount ranging from about 0.6 to about 2 g;
   threonine in a daily amount ranging from about 0.5 to about 2 g;
   tryptophan in a daily amount ranging from about 0.1 to about 0.8 g; and
   tyrosine in a daily amount ranging from about 0.5 to about 2 g.

In an embodiment the present invention relates to the kit as described above, wherein
a) said composition A comprises at least three ingredients chosen from the group consisting of:
   vitamin B1 in an amount of at least about 0.2 mg per 100 ml of liquid;
   vitamin B2 in an amount of at least about 0.2 mg per 100 ml of liquid;
   vitamin B6 in an amount of at least about 0.2 mg per 100 ml of liquid;
   zinc in an amount of at least about 1 mg per 100 ml of liquid;
   iron in an amount of at least about 2 mg per 100 ml of liquid;
   copper in an amount of at least about 0.1 mg per 100 ml of liquid;
   histidine in an amount of at least about 0.04 g per 100 ml of liquid;
   isoleucine in an amount of at least about 0.06 g per 100 ml of liquid;
   lysine in an amount of at least about 0.08 g per 100 ml of liquid;
   leucine in an amount of at least about 0.1 g per 100 ml of liquid;
   vitamin A in an amount of at least about 81 μg per 100 ml of liquid;
   folate in an amount of at least about 34 μg per 100 ml of liquid;
   magnesium in an amount of at least about 29 mg per 100 ml of liquid;
   phosphorus in an amount of at least about 30 mg per 100 ml of liquid;
   potassium in an amount of at least about 258 mg per 100 ml of liquid;
   selenium in an amount of at least about 1 μg per 100 ml pf liquid;
   3′-SL in an amount of at least about 0.8 mg per 100 ml of liquid;
   alanine in an amount of at least about 0.06 g per 100 ml of liquid;
   cystine in an amount of at least about 0.01 g per 100 ml of liquid;
   glutamic acid in an amount of at least about 0.4 g per 100 ml of liquid;
   phenylalanine in an amount of at least about 0.08 g per 100 ml of liquid;
   proline in an amount of at least about 0.1 g per 100 ml of liquid;
   serine in an amount of at least about 0.08 g per 100 ml of liquid;
   threonine in an amount of at least about 0.06 g per 100 ml of liquid;
   tyrosine in an amount of at least about 0.06 g per 100 ml of liquid; and
   valine in an amount of at least about 0.1 g per 100 ml of liquid;
b) said composition B comprises at least three ingredients chosen from the group consisting of:
   DPA in an amount of at least about 0.6 mg per 100 ml of liquid;
   ARA in an amount of at least about 0.01 g per 100 ml of liquid;
   sphingomyelin in an amount of at least about 0.6 mg per 100 ml of liquid;
   phosphatidylcholine in an amount of at least about 0.06 g per 100 ml of liquid;
   octadecatetraenoic acid in an amount of at least about 0.6 mg per 100 ml of liquid;
   oligofructose in an amount of at least about 0.2 g per 100 ml of liquid;
   6′-SL in an amount of at least about 0.6 mg per 100 ml of liquid;
   stearidonic acid in an amount of at least about 0.6 mg per 100 ml of liquid;
   beta cryptoxanthin in an amount of at least about 19 mg per 100 ml of liquid;
   alpha lactalbumin in an amount of at least about 0.06 mg per 100 ml of liquid;
   gangliosides in an amount of at least about 0.4 mg per 100 ml of liquid;
   phosphatidylinositol in an amount of at least about 0.8 mg per 100 ml of liquid; and
   3′-SL in an amount of at least about 3 mg per 100 ml of liquid;
   eicosatetraenoic acid in an amount of at least about 0.01 g per 100 ml of liquid;
c) said composition C comprises at least three ingredients chosen from the group consisting of:
   DHA in an amount of at least about 0.01 g per 100 ml of liquid;
   sphingomyelin in an amount of at least about 1 mg per 100 ml of liquid;
   phosphatidylcholine in an amount of at least about 0.08 g per 100 ml of liquid;
   histidine in an amount of at least about 0.06 g per 100 ml of liquid;
   isoleucine in an amount of at least about 0.1 g per 100 ml of liquid;
   leucine in an amount of at least about 0.2 g per 100 ml of liquid;
   valine in an amount of at least about 0.1 g per 100 ml of liquid;
   alpha carotene in an amount of at least about 43 mg per 100 ml of liquid;
   folate in an amount of at least about 51 μg per 100 ml of liquid;
   alpha lactalbumin in an amount of at least about 0.02 mg per 100 ml of liquid;
   gangliosides in an amount of at least about 0.4 mg per 100 ml of liquid;
   phosphatidylinositol in an amount of at least about 0.01 g per 100 ml of liquid;
   phosphatidylethanolamine in an amount of at least about 0.01 g per 100 ml of liquid;
   eicosenoic acid in an amount of at least about 0.02 g per 100 ml of liquid;
   stearidonic acid in an amount of at least about 0.6 mg per 100 ml of liquid;
   DPA in an amount of at least about 2 mg per 100 ml of liquid;
   3′-SL in an amount of at least about 1 mg per 100 ml of liquid;
   6′-SL in an amount of at least about 0.4 mg per 100 ml of liquid;
   alanine in an amount of at least about 0.1 g per 100 ml of liquid;
   arginine in an amount of at least about 0.1 g per 100 ml of liquid;
   aspartic acid in an amount of at least about 0.2 g per 100 ml of liquid;
   cystine in an amount of at least about 0.02 g per 100 ml of liquid;
   glutamic acid in an amount of at least about 0.4 g per 100 ml of liquid;
   glycine in an amount of at least about 0.06 g per 100 ml of liquid;
   lysine in an amount of at least about 0.1 g per 100 ml of liquid;
   methionine in an amount of at least about 0.04 g per 100 ml of liquid;
   phenylalanine in an amount of at least about 0.1 g per 100 ml of liquid;
   proline in an amount of at least about 0.2 g per 100 ml of liquid;
   serine in an amount of at least about 0.1 g per 100 ml of liquid;
   threonine in an amount of at least about 0.1 g per 100 ml of liquid;
   tryptophan in an amount of at least about 0.02 g per 100 ml of liquid;
   tyrosine in an amount of at least about 0.1 g per 100 ml of liquid;
   alpha tocopherol in an amount of at least about 0.1 mg per 100 ml of liquid;
   octadecatetraenoic acid in an amount of at least about 0.1 mg per 100 ml of liquid;
   eicosapentaenoic acid in an amount of at least about 0.02 g per 100 ml of liquid;
   phosphorus in an amount of at least about 6 mg per 100 ml of liquid;
   phosphatidylserine in an amount of at least about 0.01 g per 100 ml of liquid.

In an embodiment the present invention relates to the kit as described above, wherein
a) said composition A comprises at least three ingredients chosen from the group consisting of:
   vitamin B1 in an amount of at least about 0.2 mg per 100 ml of liquid;
   vitamin B2 in an amount of at least about 0.2 mg per 100 ml of liquid;
   vitamin B6 in an amount of at least about 0.2 mg per 100 ml of liquid;
   zinc in an amount of at least about 1 mg per 100 ml of liquid;
   iron in an amount of at least about 2 mg per 100 ml of liquid;
   copper in an amount of at least about 0.1 mg per 100 ml of liquid;
   histidine in an amount of at least about 0.04 g per 100 ml of liquid;
   isoleucine in an amount of at least about 0.06 g per 100 ml of liquid;
   lysine in an amount of at least about 0.08 g per 100 ml of liquid;
   leucine in an amount of at least about 0.1 g per 100 ml of liquid;
   vitamin A in an amount of at least about 81 μg per 100 ml of liquid;
   folate in an amount of at least about 34 μg per 100 ml of liquid;
   magnesium in an amount of at least about 29 mg per 100 ml of liquid;
   phosphorus in an amount of at least about 149 mg per 100 ml of liquid;
   potassium in an amount of at least about 258 mg per 100 ml of liquid;
   selenium in an amount of at least about 10 mg per 100 ml pf liquid;
   3′-SL in an amount of at least about 0.8 mg per 100 ml of liquid;
   alanine in an amount of at least about 0.06 g per 100 ml of liquid;
   cystine in an amount of at least about 0.01 g per 100 ml of liquid;
   glutamic acid in an amount of at least about 0.4 g per 100 ml of liquid;
   phenylalanine in an amount of at least about 0.08 g per 100 ml of liquid;
   proline in an amount of at least about 0.1 g per 100 ml of liquid;
   serine in an amount of at least about 0.08 g per 100 ml of liquid;
   threonine in an amount of at least about 0.06 g per 100 ml of liquid;
   tyrosine in an amount of at least about 0.06 g per 100 ml of liquid; and
   valine in an amount of at least about 0.1 g per 100 ml of liquid;
b) said composition B comprises at least three ingredients chosen from the group consisting of:
   DPA in an amount of at least about 0.6 mg per 100 ml of liquid;
   ARA in an amount of at least about 0.01 g per 100 ml of liquid;
   sphingomyelin in an amount of at least about 0.6 mg per 100 ml of liquid;
   phosphatidylcholine in an amount of at least about 0.06 g per 100 ml of liquid;
   octadecatetraenoic acid in an amount of at least about 0.6 mg per 100 ml of liquid;
   oligofructose in an amount of at least about 0.2 g per 100 ml of liquid;
   6′-SL in an amount of at least about 0.6 mg per 100 ml of liquid;
   stearidonic acid in an amount of at least about 0.6 mg per 100 ml of liquid;
   beta cryptoxanthin in an amount of at least about 19 mg per 100 ml of liquid;
   alpha lactalbumin in an amount of at least about 0.06 mg per 100 ml of liquid;
   gangliosides in an amount of at least about 0.4 mg per 100 ml of liquid;
   phosphatidylinositol in an amount of at least about 0.8 mg per 100 ml of liquid; and
   3′-SL in an amount of at least about 3 mg per 100 ml of liquid;
c) said composition C comprises at least three ingredients chosen from the group consisting of:
   DHA in an amount of at least about 0.01 g per 100 ml of liquid;
   sphingomyelin in an amount of at least about 1 mg per 100 ml of liquid;
   phosphatidylcholine in an amount of at least about 0.08 g per 100 ml of liquid;
   histidine in an amount of at least about 0.06 g per 100 ml of liquid;
   isoleucine in an amount of at least about 0.1 g per 100 ml of liquid;
   leucine in an amount of at least about 0.2 g per 100 ml of liquid;
   valine in an amount of at least about 0.1 g per 100 ml of liquid;
   alpha carotene in an amount of at least about 43 mg per 100 ml of liquid;
   folate in an amount of at least about 51 μg per 100 ml of liquid;
   alpha lactalbumin in an amount of at least about 0.02 mg per 100 ml of liquid;
   gangliosides in an amount of at least about 0.4 mg per 100 ml of liquid;
   phosphatidylinositol in an amount of at least about 0.01 g per 100 ml of liquid;
   phosphatidylethanolamine in an amount of at least about 0.01 g per 100 ml of liquid;
   eicosenoic acid in an amount of at least about 0.02 g per 100 ml of liquid;
   stearidonic acid in an amount of at least about 0.6 mg per 100 ml of liquid;
   DPA in an amount of at least about 2 mg per 100 ml of liquid;
   3′-SL in an amount of at least about 1 mg per 100 ml of liquid;
   6′-SL in an amount of at least about 0.4 mg per 100 ml of liquid;
   alanine in an amount of at least about 0.1 g per 100 ml of liquid;
arginine in an amount of at least about 0.1 g per 100 ml of liquid;
aspartic acid in an amount of at least about 0.2 g per 100 ml of liquid;
cystine in an amount of at least about 0.02 g per 100 ml of liquid;
glutamic acid in an amount of at least about 0.4 g per 100 ml of liquid;
glycine in an amount of at least about 0.06 g per 100 ml of liquid;
lysine in an amount of at least about 0.1 g per 100 ml of liquid;
methionine in an amount of at least about 0.04 g per 100 ml of liquid;
phenylalanine in an amount of at least about 0.1 g per 100 ml of liquid;
proline in an amount of at least about 0.2 g per 100 ml of liquid;
serine in an amount of at least about 0.1 g per 100 ml of liquid;
threonine in an amount of at least about 0.1 g per 100 ml of liquid;
tryptophan in an amount of at least about 0.02 g per 100 ml of liquid; and
tyrosine in an amount of at least about 0.1 g per 100 ml of liquid.

In an embodiment, the present invention relates to the kit as described above wherein
a) said composition A comprises at least three ingredients chosen from the group consisting of:
   vitamin B1 in an amount ranging from about 0.2 to about 0.4 mg per 100 ml of liquid;
   vitamin B2 in an amount ranging from about 0.2 to about 0.6 mg per 100 ml of liquid;
   vitamin B6 in an amount ranging from about 0.2 to about 1 mg per 100 ml of liquid;
   zinc in an amount ranging from about 1 to about 2.5 mg per 100 ml of liquid;
   iron in an amount ranging from about 2 to about 6 mg per 100 ml of liquid;
   copper in an amount ranging from about 0.1 to about 0.2 mg per 100 ml of liquid;
   histidine in an amount ranging from about 0.04 to about 0.2 g per 100 ml of liquid;
   isoleucine in an amount ranging from about 0.06 to about 0.4 g per 100 ml of liquid;
   lysine in an amount ranging from about 0.08 to about 0.6 g per 100 ml of liquid;
   leucine in an amount ranging from about 0.1 to about 0.8 g per 100 ml of liquid;
   vitamin A in an amount ranging from about 81 to about 500 μg per 100 ml of liquid;
   folate in an amount ranging from about 34 to about 95 μg per 100 ml of liquid;
   magnesium in an amount ranging from about 29 to about 75 mg per 100 ml of liquid;
   phosphorus in an amount ranging from about 33 to about 343 mg per 100 ml of liquid;
   potassium in an amount ranging from about 258 to about 830 mg per 100 ml of liquid;
   selenium in an amount ranging from about 10 to about 31 μg per 100 ml pf liquid;
   3′-SL in an amount ranging from about 0.8 to about 14 mg per 100 ml of liquid;
   alanine in an amount ranging from about 0.06 to about 0.4 g per 100 ml of liquid;
   cystine in an amount ranging from about 0.01 to about 0.08 g per 100 ml of liquid;
   glutamic acid in an amount ranging from about 0.4 to about 2 g per 100 ml of liquid;
   phenylalanine in an amount ranging from about 0.08 to about 0.4 g per 100 ml of liquid;
   proline in an amount ranging from about 0.1 to about 0.6 g per 100 ml of liquid;
   serine in an amount ranging from about 0.08 to about 0.4 g per 100 ml of liquid;
   threonine in an amount ranging from about 0.06 to about 0.4 g per 100 ml of liquid;
   tyrosine in an amount ranging from about 0.06 to about 0.4 g per 100 ml of liquid; and
   valine in an amount ranging from about 0.1 to about 0.4 g of per 100 ml of liquid;
b) said composition B comprises at least three ingredients chosen from the group consisting of:
   DPA in an amount ranging from about 0.6 to about 11 mg per 100 ml of liquid;
   ARA in an amount ranging from about 0.01 to about 0.08 g per 100 ml of liquid;
   sphingomyelin in an amount ranging from about 0.6 to about 11 mg per 100 ml of liquid;
   phosphatidylcholine in an amount ranging from about 0.06 to about 1 g per 100 ml of liquid;
   octadecatetraenoic acid in an amount ranging from about 0.6 to about 7 mg per 100 ml of liquid;
   oligofructose in an amount ranging from about 0.2 to about 2 g per 100 ml of liquid;
   6′-SL in an amount ranging from about 0.6 to about 5 mg per 100 ml of liquid;
   stearidonic acid in an amount ranging from about 0.6 to about 9 mg per 100 ml of liquid;
   beta cryptoxanthin in an amount ranging from about 19 to about 98 mg per 100 ml of liquid;
   alpha lactalbumin in an amount ranging from about 0.06 to about 0.2 mg per 100 ml of liquid;
   gangliosides in an amount ranging from about 0.4 to about 3 mg per 100 ml of liquid;
   phosphatidylinositol in an amount ranging from about 0.8 to about 12 mg per 100 ml of liquid; and
   3′-SL in an amount ranging from about 3 to about 23 mg per 100 ml of liquid;
   eicosatetraenoic acid in an amount ranging from about 0.01 to about 0.06 g per 100 ml of liquid;
c) said composition C comprises at least three ingredients chosen from the group consisting of:
   DHA in an amount ranging from about 0.01 to about 0.06 g per 100 ml of liquid;
   sphingomyelin in an amount ranging from about 1 to about 20 mg per 100 ml of liquid;
   phosphatidylcholine in an amount ranging from about 0.08 to about 0.8 g per 100 ml of liquid;
   histidine in an amount ranging from about 0.06 to about 0.2 g per 100 ml of liquid;
   isoleucine in an amount ranging from about 0.1 to about 0.4 g per 100 ml of liquid;
   leucine in an amount ranging from about 0.2 to about 0.8 g per 100 ml of liquid;
   valine in an amount ranging from about 0.1 to about 0.6 g per 100 ml of liquid;
   alpha carotene in an amount ranging from about 43 to about 424 mg per 100 ml of liquid;
   folate in an amount ranging from about 51 to about 82 μg per 100 ml of liquid;
   alpha lactalbumin in an amount ranging from about 0.02 to about 0.2 mg per 100 ml of liquid;
   gangliosides in an amount ranging from about 0.4 to about 3 mg per 100 ml of liquid;
   phosphatidylinositol in an amount ranging from about 0.01 to about 0.04 g per 100 ml of liquid;
   phosphatidylethanolamine in an amount ranging from about 0.01 to about 0.2 g per 100 ml of liquid;
   eicosenoic acid in an amount ranging from about 0.01 to about 0.12 g per 100 ml of liquid;
   stearidonic acid in an amount ranging from about 0.6 to about 14 mg per 100 ml of liquid;
   DPA in an amount ranging from about 2 to about 9 mg per 100 ml of liquid;
   3′-SL in an amount ranging from about 1 to about 21 mg per 100 ml of liquid;
   6′-SL in an amount ranging from about 0.4 to about 4 mg per 100 ml of liquid;
   alanine in an amount ranging from about 0.1 to about 0.4 g per 100 ml of liquid;
   arginine in an amount ranging from about 0.1 to about 0.4 g per 100 ml of liquid;
   aspartic acid in an amount ranging from about 0.2 to about 0.8 g per 100 ml of liquid;
   cystine in an amount ranging from about 0.02 to about 0.1 g per 100 ml of liquid;
   glutamic acid in an amount ranging from about 0.4 to about 2 g per 100 ml of liquid;
   glycine in an amount ranging from about 0.06 to about 0.4 g per 100 ml of liquid;
   lysine in an amount ranging from about 0.1 to about 0.6 g per 100 ml of liquid;
   methionine in an amount ranging from about 0.04 to about 0.2 g per 100 ml of liquid;
   phenylalanine in an amount ranging from about 0.1 to about 0.4 g per 100 ml of liquid;
   proline in an amount ranging from about 0.2 to about 0.8 g per 100 ml of liquid;
   serine in an amount ranging from about 0.1 to about 0.6 g per 100 ml of liquid;
   threonine in an amount ranging from about 0.1 to about 0.4 g per 100 ml of liquid;
   tryptophan in an amount ranging from about 0.02 to about 0.2 g per 100 ml of liquid; and
   tyrosine in an amount ranging from about 0.1 to about 0.4 g per 100 ml of liquid;
   alpha tocopherol in an amount ranging from about 0.1 to about 3.6 g per 100 ml of liquid;
   octadecatetraenoic acid in an amount ranging from about 0.1 to about 11.4 mg per 100 ml of liquid;
   eicosapentaenoic acid in an amount ranging from about 0.1 to about 26 mg per 100 ml of liquid;
   phosphorus in an amount ranging from about 6 to about 300 mg per 100 ml of liquid;
   phosphatidylserine in an amount ranging from about 0.01 to about 0.4 g per 100 ml of liquid.

In an embodiment, the present invention relates to the kit as described above wherein
a) said composition A comprises at least three ingredients chosen from the group consisting of:
   vitamin B1 in an amount ranging from about 0.2 to about 0.4 mg per 100 ml of liquid;
   vitamin B2 in an amount ranging from about 0.2 to about 0.6 mg per 100 ml of liquid;
   vitamin B6 in an amount ranging from about 0.2 to about 1 mg per 100 ml of liquid;
   zinc in an amount ranging from about 1 to about 2 mg per 100 ml of liquid;
   iron in an amount ranging from about 2 to about 6 mg per 100 ml of liquid;
   copper in an amount ranging from about 0.1 to about 0.2 mg per 100 ml of liquid;
   histidine in an amount ranging from about 0.04 to about 0.2 g per 100 ml of liquid;
   isoleucine in an amount ranging from about 0.06 to about 0.4 g per 100 ml of liquid;
   lysine in an amount ranging from about 0.08 to about 0.6 g per 100 ml of liquid;
   leucine in an amount ranging from about 0.1 to about 0.8 g per 100 ml of liquid;
   vitamin A in an amount ranging from about 81 to about 184 μg per 100 ml of liquid;
   folate in an amount ranging from about 34 to about 61 μg per 100 ml of liquid;
   magnesium in an amount ranging from about 29 to about 75 mg per 100 ml of liquid;
   phosphorus in an amount ranging from about 149 to about 343 mg per 100 ml of liquid;
   potassium in an amount ranging from about 258 to about 830 mg per 100 ml of liquid;
   selenium in an amount ranging from about 10 to about 18 mg per 100 ml pf liquid;
   3′-SL in an amount ranging from about 0.8 to about 14 mg per 100 ml of liquid;
   alanine in an amount ranging from about 0.06 to about 0.4 g per 100 ml of liquid;
   cystine in an amount ranging from about 0.01 to about 0.08 g per 100 ml of liquid;
   glutamic acid in an amount ranging from about 0.4 to about 2 g per 100 ml of liquid;
   phenylalanine in an amount ranging from about 0.08 to about 0.4 g per 100 ml of liquid;
   proline in an amount ranging from about 0.1 to about 0.6 g per 100 ml of liquid;
   serine in an amount ranging from about 0.08 to about 0.4 g per 100 ml of liquid;
   threonine in an amount ranging from about 0.06 to about 0.4 g per 100 ml of liquid;
   tyrosine in an amount ranging from about 0.06 to about 0.4 g per 100 ml of liquid; and
   valine in an amount ranging from about 0.1 to about 0.4 g of per 100 ml of liquid;
b) said composition B comprises at least three ingredients chosen from the group consisting of:
   DPA in an amount ranging from about 0.6 to about 11 mg per 100 ml of liquid;
   ARA in an amount ranging from about 0.01 to about 0.08 g per 100 ml of liquid;
   sphingomyelin in an amount ranging from about 0.6 to about 11 mg per 100 ml of liquid;
   phosphatidylcholine in an amount ranging from about 0.06 to about 1 g per 100 ml of liquid;
   octadecatetraenoic acid in an amount ranging from about 0.6 to about 7 mg per 100 ml of liquid;
   oligofructose in an amount ranging from about 0.2 to about 2 g per 100 ml of liquid;
   6′-SL in an amount ranging from about 0.6 to about 5 mg per 100 ml of liquid;
   stearidonic acid in an amount ranging from about 0.6 to about 9 mg per 100 ml of liquid;
   beta cryptoxanthin in an amount ranging from about 19 to about 98 mg per 100 ml of liquid;
   alpha lactalbumin in an amount ranging from about 0.06 to about 0.2 mg per 100 ml of liquid;
   gangliosides in an amount ranging from about 0.4 to about 3 mg per 100 ml of liquid;
   phosphatidylinositol in an amount ranging from about 0.8 to about 12 mg per 100 ml of liquid; and
   3′-SL in an amount ranging from about 3 to about 23 mg per 100 ml of liquid;
c) said composition C comprises at least three ingredients chosen from the group consisting of:
   DHA in an amount ranging from about 0.01 to about 0.06 g per 100 ml of liquid;
   sphingomyelin in an amount ranging from about 1 to about 20 mg per 100 ml of liquid;
   phosphatidylcholine in an amount ranging from about 0.08 to about 0.8 g per 100 ml of liquid;
   histidine in an amount ranging from about 0.06 to about 0.2 g per 100 ml of liquid;
   isoleucine in an amount ranging from about 0.1 to about 0.4 g per 100 ml of liquid;
   leucine in an amount ranging from about 0.2 to about 0.8 g per 100 ml of liquid;
   valine in an amount ranging from about 0.1 to about 0.6 g per 100 ml of liquid;
   alpha carotene in an amount ranging from about 43 to about 424 mg per 100 ml of liquid;
   folate in an amount ranging from about 51 to about 82 μg per 100 ml of liquid;
   alpha lactalbumin in an amount ranging from about 0.02 to about 0.2 mg per 100 ml of liquid;
   gangliosides in an amount ranging from about 0.4 to about 3 mg per 100 ml of liquid;
   phosphatidylinositol in an amount ranging from about 0.01 to about 0.04 g per 100 ml of liquid;
   phosphatidylethanolamine in an amount ranging from about 0.01 to about 0.2 g per 100 ml of liquid;
   eicosenoic acid in an amount ranging from about 0.02 to about 0.1 g per 100 ml of liquid;
   stearidonic acid in an amount ranging from about 0.6 to about 14 mg per 100 ml of liquid;
   DPA in an amount ranging from about 2 to about 9 mg per 100 ml of liquid;
   3′-SL in an amount ranging from about 1 to about 21 mg per 100 ml of liquid;
   6′-SL in an amount ranging from about 0.4 to about 4 mg per 100 ml of liquid;
   alanine in an amount ranging from about 0.1 to about 0.4 g per 100 ml of liquid;
   arginine in an amount ranging from about 0.1 to about 0.4 g per 100 ml of liquid;
   aspartic acid in an amount ranging from about 0.2 to about 0.8 g per 100 ml of liquid;
   cystine in an amount ranging from about 0.02 to about 0.1 g per 100 ml of liquid;
   glutamic acid in an amount ranging from about 0.4 to about 2 g per 100 ml of liquid;
   glycine in an amount ranging from about 0.06 to about 0.4 g per 100 ml of liquid;
   lysine in an amount ranging from about 0.1 to about 0.6 g per 100 ml of liquid;
   methionine in an amount ranging from about 0.04 to about 0.2 g per 100 ml of liquid;
   phenylalanine in an amount ranging from about 0.1 to about 0.4 g per 100 ml of liquid;
   proline in an amount ranging from about 0.2 to about 0.8 g per 100 ml of liquid;
   serine in an amount ranging from about 0.1 to about 0.6 g per 100 ml of liquid;
   threonine in an amount ranging from about 0.1 to about 0.4 g per 100 ml of liquid;
   tryptophan in an amount ranging from about 0.02 to about 0.2 g per 100 ml of liquid; and
   tyrosine in an amount ranging from about 0.1 to about 0.4 g per 100 ml of liquid.

In one embodiment, the present invention relates to the kit as described above wherein each of said compositions comprise at least four said ingredients chosen from said group.

In one embodiment, the present invention relates to the kit as described above wherein each of said compositions comprise at least five said ingredients chosen from said group.

In one embodiment, the present invention relates to the kit as described above wherein each of said compositions comprise at least six said ingredients chosen from said group.

Suitably, each composition of the kit according to the present invention can be different from the other compositions, such that composition A can be different from compositions B and C, composition B can be different from compositions A and C and composition C can be different from compositions A and B in at least one comprised ingredient or in in at least two comprised ingredients, or in at least three comprised ingredients or in at least four comprised ingredients.

In one embodiment, the present invention relates to the kit as described above wherein each of said compositions differ one from the other, in at least one comprised ingredient, or in in at least two comprised ingredients, or in at least three comprised ingredients or in at least four comprised ingredients.

In one embodiment, the composition A comprised in the kit according to the present invention can differ from composition B and/or C in that it comprises at least one ingredient, at least two ingredients, or at least three ingredients, or at least four ingredients that is/are not comprised in composition B and/or C.

In one embodiment, the composition B comprised in the kit according to the present invention can differ from composition A and/or C in that it comprises at least one ingredient, at least two ingredients, or at least three ingredients, or at least four ingredients that is/are not comprised in composition A and/or C.

In one embodiment, the composition C comprised in the kit according to the present invention can differ from composition A and/or B in that it comprises at least one ingredient, at least two ingredients, or at least three ingredients, or at least four ingredients that is/are not comprised in composition A and/or B.

Advantageously, the kit according to the present invention promoted myelination of targeted brain regions, in particular the myelination of social brain that is associated with socio-emotional development. The kit as claimed has the advantage of targeting specific brain regions involved in socio-emotional development, in particular social brain, and at specific age windows following the dynamic needs of the developing brain.

In one embodiment, the present invention relates to the kit as described above wherein the targeted brain region is at least one region of the social brain. In another embodiment, the present invention relates to the kit as described above wherein the targeted brain region is at least one region of the social brain associated with socio-emotional development.

In one embodiment, the present invention relates to the kit as described above wherein the young individual is a human, a pet, an infant, a young child or a child.

In one embodiment, the present invention relates to a method for promoting brain myelination of targeted brain regions of a young individual comprising sequential administering to a young individual the kit as described above.

In one embodiment, the present invention relates to the method as described above wherein the targeted brain region is at least one region of the social brain.

In one embodiment, the present invention relates to the method as described above wherein the targeted brain region is at least one region of the social brain associated with socio-emotional development.

In one embodiment, the present invention relates to a staged feeding regimen of a young individual comprising the steps of
a) administering the composition A according to the present invention at age from 0 to 20 months;
b) administering the composition B according to the present invention at age from 20 to 30 months;
c) administering the composition C according to the present invention at age from 30 to 66 months.

In one embodiment, the present invention relates to the staged feeding regimen of a young individual as described above comprising the steps of
a) administering the composition A according to the present invention at age from 6 to 20 months;
b) administering the composition B according to the present invention at age from 20 to 30 months;
c) administering the composition C according to the present invention at age from 30 to 66 months.

In one embodiment, the present invention relates to the staged feeding regimen of a young individual as described above comprising the steps of
a) administering the composition A according to the present invention at age from 6 to 20 months;
b) administering the composition B according to the present invention at age from 20 to 30 months;
c) administering the composition C according to the present invention at age from 30 to 60 months.

In one embodiment, the present invention relates to the staged feeding regimen of a young individual as described above comprising the steps of
a) administering the composition A according to the present invention at age from 0 to 20 months;
b) administering the composition B according to the present invention at age from 20 to 30 months;
c) administering the composition C according to the present invention at age from 30 to 60 months.

In one embodiment, the present invention relates to a use of the staged feeding regimen as described above to promote myelination of targeted brain regions.

In one embodiment, the present invention relates to the use of the stage feeding regimen wherein the targeted brain region is at least one region of the social brain. In another embodiment, the present invention related to the use of the staged feeding regiment wherein the targeted brain region is at least one region of the social brain associated with socio-emotional development.

### Compositions of the Kit

Unless otherwise indicated, all amounts indicated for nutrients are expressed as amounts per weight in a daily amount or per 100 ml of liquid.

Where a nutrient may be comprised in a composition under different forms (as such or in the form of salts, complexes or more complex structures comprising the nutrient) the amounts reported hereafter are to be intended to refer to the amount of the nutrient as such.

Minerals such as but not limited to zinc, iron, copper, magnesium, phosphorus, potassium, selenium, calcium, and combinations thereof are usually added to the compositions of the invention in salt form.

Phosphorus may be incorporated in the composition of the invention as such or in the form of a physiologically acceptable salt and/or via any source comprising phosphorus for example: calcium phosphate, calcium hydrogen phosphate.

Calcium may be incorporated in the composition of the invention as such or in the form of a physiologically acceptable salt and/or via any source comprising calcium. For example, calcium carbonate, calcium chloride, calcium salts of citric acid, calcium gluconate, calcium glycerophosphate, calcium lactate, calcium hydroxide, calcium salts of orthophosphoric acid.

Vitamin B12 may be incorporated in the nutritional compositions of the kit of the invention as such or in the form of a physiologically acceptable salt thereof or mixtures thereof, or via any source comprising vitamin B12. In particular vitamin B12 may be incorporated into the composition in its pure form, as cyanocobalamin, hydroxocobalamin, and any combination thereof.

Folic acid may be present in the compositions of the kit either as such or in the form of one physiologically acceptable salt thereof (folate) and mixtures thereof.

Amino acids comprised in the compositions, such as but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine, and mixtures thereof, may be naturally occurring or synthesized.

Fatty acids in the compositions, such as ARA, DHA, DPA, eicosenoic acid, stearidonic acid, come from natural sources, non-limiting examples of which include, eggs, algae, fish oil, mould, yeast, seeds, plants e.g., soy, and animal sources e.g., bovine brains, and/or mammalian milk or extracts thereof. Non limiting examples of soy sources include soy lecithin-food additive, non-limiting examples of mammalian milk include bovine, camel, sheep, goat milk including skilled milks. Non limiting extracts of milk include protein extracts, milk fat globule membranes (MFGM) and extracts comprising them. Fatty acid derivatives may also come from palm oil, tallow, lard, cotton seed oil, peanut oil.

These fatty acids may be an important component of myelin enabling it to wrap around and enrobe axons.

The origin of DHA, ARA, DPA may be of importance in the context of the present invention as the mentioned fatty acids obtained from fish oil may have a different effect compared to microbial ones.

The phospholipids, such as sphingomyelin, phosphatidylcholine, phosphatidylserine, phosphatidylinositol, phosphatidylethanolamine, comprised in the composition of the invention may be natural, synthetic or a mixture thereof. They may be used in the composition of the invention in their pure form, or substantially pure form. Alternatively, they may be added in the form of a source comprising them.

In particular at least one of the mentioned phospholipids comes from natural sources, non-limiting examples of which include, eggs, soy, bovine brains, and/or mammalian milk or extracts thereof. Non limiting examples of soy sources include soy lecithin-food additive, non-limiting examples of mammalian milk include bovine, camel, sheep, goat milk including skilled milks. Non limiting extracts of milk include protein extracts e.g. whey protein and casein, milk fat globule membranes (MFGM) and extracts comprising them.

Sphingomyelin may be synthesized from ceramide and/or one or more ganglioside and phosphatidylcholine, accordingly, it may be particularly beneficial if ceramide and/or one or more ganglioside is used in combination with phosphatidylcholine a metabolic precursor or metabolite thereof.

Within the context of the present invention, sphingomyelin may be administered as such or in the form of metabolic precursors and/or a metabolite thereof. Without wishing to be bound by theory, it is considered that such metabolic precursors and/or metabolites elicit the same beneficial effect in promoting myelination in the brain regions as the direct administration of sphingomyelin does. Non limiting exemplary species in this respect are: galactoceramides, glucoceramides, sphingosine, sphingosine-l-phosphate, ceramide, D-erythro-dihydroceramide and ceramide-1-phosphate and gangliosides.

Sphingomyelin is present in natural sources such as: alpha-lactalbumin, egg, bovine brain, or bovine milk etc.

A sphingomyelin and/or metabolic precursors or metabolites thereof may be incorporated in the nutritional compositions of the invention as a single species, as an ingredient consisting of a mixture of different sphingomyelin species or by addition of a natural or synthetic ingredient comprising one or more sphingomyelin species. Sphingomyelin may be incorporated in the nutritional compositions of the invention as comprised in skim milk powder, alpha lactalbumin, whey protein concentrate and/or whey protein concentrate enriched in alpha lactalbumin.

### Composition A

As mentioned before, it is beneficial that the composition A of the kit to be administrated to a young individual at age from 0 to 20 months, preferably from 6 to 20 months, as it comprises targeted and appropriate nutrients that are proved to promote myelination of targeted brain regions, in particular of the social brain, at this specific age following specific stage of brain development. It therefore avoids the delivery of the nutrients that may have no effect to this process or may even cause negative effect such as sub-optimal myelination leading to impaired or delayed brain development.

Suitably, the composition A is administered to a young individual at age from 0 to 20 months, for example from 1 to 20 months, from 2 to 20 months, from 3 to 20 months, from 4 to 20 months, from 5 to 20 months or from 6 to 20 months.

Composition A is advantageously tailored to be administered to a young individual in myelin-nutrient window 1, to effectively promote myelination in targeted brain region, in particular social brain associated with socio-emotional development, at the stage where a steep and fast increase of myelination is observed.

In one embodiment of the invention composition A comprises at least three ingredients chosen from the group consisting of vitamin B1, vitamin B2, vitamin B6, zinc, iron, copper, histidine, isoleucine, lysine, leucine, vitamin A, folate, magnesium, phosphorus, potassium, selenium, 3′-SL, alanine, cystine, glutamic acid, phenylalanine, proline, serine, threonine, tyrosine, valine.

In a preferred embodiment, said composition A comprises at least three ingredients chosen from the group consisting of vitamin B1, vitamin B2, vitamin B6, zinc, iron, copper, histidine, isoleucine, lysine, leucine.

The composition A of the present invention may include one or more of the following vitamins or derivations thereof:
vitamin B1 in a daily amount of at least about 0.8 mg, preferably in a daily amount ranging from about 0.8 to about 2 mg, or in an amount of at least about 0.2 mg per 100 ml of liquid, preferably in an amount ranging from about 0.2 to about 0.4 mg per 100 ml of liquid, as thiamin, thiamin pyrophosphate, TPP, thiamin triphosphate, TTP, thiamin hydrochloride, thiamin mononitrate;
vitamin B2 in a daily amount of at least about 1 mg, preferably in a daily amount ranging from about 1 to about 3 mg, or in an amount of at least about 0.2 mg per 100 ml of liquid, preferably in an amount ranging from about 0.2 to about 0.6 mg per 100 ml of liquid, as riboflavin, flavin mononucleotide, FMN, flavin adenine dinucleotide, FAD, lactoflavin, ovoflavin;
vitamin B6 in a daily amount of at least about 0.9 mg, preferably in a daily amount ranging from about 0.9 to about 5 mg, or in an amount of at least about 0.2 mg per 100 ml of liquid, preferably in an amount ranging from about 0.2 to about 1 mg per 100 ml of liquid, as pyridoxine, pyridoxal, pyridoxamine, pyridoxine hydrochloride;
vitamin A in a daily amount of at least about 400 μg, preferably in a daily amount ranging from about 400 to about 2600 μg, or from about 400 to about 900 μg, or in an amount of at least about 81 μg per 100 ml of liquid, preferably in an amount ranging from about 81 to about 540 μg per 100 ml of liquid, or in an amount ranging from about 81 to about 184 μg per 100 ml of liquid as retinol, retinyl acetate, retinyl palmitate, retinyl esters with other long-chain fatty acids, retinal, retinoic acid, retinol esters;
folate in a daily amount of at least about 168 μg, preferably in a daily amount ranging from about 168 to about 460 μg, or in a daily amount ranging from about 168 to about 300 μg, or in an amount of at least about 34 μg per 100 ml of liquid, preferably in an amount ranging from about 34 to about 95 μg per 100 ml of liquid, or in an amount ranging from about 34 to about 61 μg per 100 ml of liquid, as folic acid, folacin, pteroylglutamic acid.

Zinc may be incorporated in the composition A of the invention in a daily amount of at least 6 mg, preferably in a daily amount ranging from about 6 to about 13 mg, or in a daily amount ranging from about 6 to about 7.5 mg, or in an amount of at least about 1 mg per 100 ml of liquid, preferably in an amount ranging from about 1 to about 2.6 mg per 100 ml, or in an amount ranging from about 1 to about 2 mg per 100 ml of liquid, in the form of a physiologically acceptable salt such as, for example: zinc nitrate, zinc sulfate, zinc gluconate, zinc acetate or mixtures thereof, or in the form of a physiologically acceptable zinc complex (such as for example zinc picolinate) or mixtures thereof.

Iron may be incorporated in the composition A of the invention in a daily amount of at least 8 mg, preferably in a daily amount ranging from about 8 to about 29 mg, or in an amount of at least about 2 mg per 100 ml of liquid, preferably in an amount ranging from about 2 to about 6 mg per 100 ml of liquid, either in free form, or in the form of a physiologically acceptable salt such as, for example: ferric citrate, ferric phosphate, ferric pyrophosphate, ferrous ascorbate, ferrous carbonate, ferrous citrate, ferrous fumarate, ferrous gluconate, ferrous lactate, ferrous sulfate or mixtures thereof or in the form of one physiologically acceptable iron complex such as for example EDTA ferric sodium salt, and mixtures thereof.

Copper may be incorporated in the composition A of the invention in a daily amount of at least about 0.6 mg, preferably in a daily amount ranging from about 0.6 to about 1.1 mg, or in an amount of at least about 0.1 mg per 100 ml of liquid, preferably in an amount ranging from about 0.1 to about 0.2 mg per 100 ml of liquid, as such or in the form of a physiologically acceptable salt and/or via any source comprising copper. For example, copper may be incorporated into the composition as: copper sulfate and/or copper gluconate and/or copper carbonate, and/or copper citrate, and/or copper-lysine complex.

Magnesium may be incorporated in the composition A of the invention in a daily amount of at least about 143 mg, preferably in a daily amount ranging from about 143 to about 369 mg, or in an amount of at least about 29 mg per 100 ml of liquid, preferably in an amount ranging from about 29 to about 75 mg per 100 ml of liquid, as such or in the form of a physiologically acceptable salt and/or via any source comprising magnesium. For example, magnesium carbonate, magnesium chloride, magnesium oxide, magnesium sulphate, magnesium gluconate, magnesium hydroxide, magnesium salts of citric acid, magnesium salts of orthophosphoric acid.

The composition A of the invention may include one or more of the following amino acids in the following amounts:
histidine in a daily amount of at least about 0.2 g, preferably in a daily amount ranging from about 0.2 to about 1 g, or in an amount of at least about 0.04 g per 100 ml of liquid, preferably in an amount ranging from about 0.04 to about 0.2 g per 100 ml of liquid;
isoleucine in a daily amount of at least about 0.3 g, preferably in a daily amount ranging from about 0.3 to about 2 g, or in an amount of at least about 0.06 g per 100 ml of liquid, preferably in an amount ranging from about 0.06 to about 0.4 g per 100 ml of liquid;
lysine in a daily amount of at least about 0.4 g, preferably in a daily amount ranging from about 0.4 to about 3 g, or in an amount of at least about 0.08 g per 100 ml of liquid, preferably in an amount ranging from about 0.08 to about 0.6 g per 100 ml of liquid;
leucine in a daily amount of at least 0.6 g, preferably in a daily amount ranging from about 0.6 to about 4 g, or in an amount of at least about 0.1 g per 100 ml of liquid, preferably in an amount ranging from about 0.1 to about 0.8 g per 100 ml of liquid;
alanine in a daily amount of at least about 0.3 g, preferably in a daily amount ranging from about 0.3 to about 2 g, or in an amount of at least about 0.06 g per 100 ml of liquid, preferably in an amount ranging from about 0.06 to about 0.4 g per 100 ml of liquid;
cystine in a daily amount of at least about 0.07 g, preferably in a daily amount ranging from about 0.07 to about 0.4 g, or in an amount of at least about 0.01 g per 100 ml of liquid, preferably in an amount ranging from about 0.01 to about 0.08 g per 100 ml of liquid;
glutamic acid in a daily amount of at least about 2 g, preferably in a daily amount ranging from about 2 to about 10 g, or in an amount of at least about 0.4 g per 100 ml of liquid, preferably in an amount ranging from about 0.4 to about 2 g per 100 ml of liquid;
phenylalanine in a daily amount of at least about 0.4 g, preferably in a daily amount ranging from about 0.4 to about 2 g, or in an amount of at least about 0.08 g per 100 ml of liquid, preferably in an amount ranging from about 0.08 to about 0.4 g per 100 ml of liquid;
proline in a daily amount of at least about 0.6 g, preferably in a daily amount ranging from about 0.6 to about 3 g, or in an amount of at least 0.1 g per 100 ml of liquid, preferably in an amount ranging from about 0.1 to about 0.6 g per 100 ml of liquid;
serine in a daily amount of at least about 0.4 g, preferably in a daily amount ranging from about 0.4 to about 2 g, or in an amount of at least 0.08 g per 100 ml of liquid, preferably in an amount ranging from about 0.08 to about 0.4 g per 100 ml of liquid;
threonine in a daily amount of at least about 0.3 g, preferably in a daily amount ranging from about 0.3 g to about 2 g, or in an amount of at least about 0.06 g per 100 ml of liquid, preferably in an amount ranging from about 0.06 to about 0.4 g per 100 ml of liquid;
tyrosine in a daily amount of at least about 0.3 g, preferably in a daily amount ranging from about 0.3 to about 2 g, or in an amount of at least about 0.06 g per 100 ml of liquid, preferably in an amount ranging from about 0.06 to about 0.4 g per 100 ml of liquid;
valine in a daily amount of at least about 0.5 g, preferably in a daily amount ranging from about 0.5 to about 2 g, or in an amount of at least about 0.1 g per 100 ml of liquid, preferably in an amount ranging from about 0.1 to about 0.4 g of per 100 ml of liquid;

The composition A may also comprise at least one of the following in the following amount:
phosphorus in a daily amount preferably of at least about 688 mg, or of at least about 731 mg, preferably in a daily amount ranging from about 688 to about 1687 mg, or from about 731 to about 1687 mg, or in an amount of at least about 141 mg per 100 ml of liquid, preferably in an amount ranging from about 141 to about 343 mg per 100 ml of liquid;
potassium in a daily amount of at least about 1267 mg, preferably in a daily amount ranging from about 1267 to about 4085 mg, or in an amount of at least 258 mg per 100 ml of liquid, preferably in an amount ranging from about 258 to about 830 mg per 100 ml of liquid;
selenium in a daily amount of at least about 51 μg, preferably in a daily amount ranging from about 51 to about 150 μg, or from about 51 to about 90 μg, or in an amount of at least about 10 mg per 100 ml pf liquid, preferably in an amount ranging from about 10 to about 31 μg per 100 ml pf liquid;
3′-SL in a daily amount of at least about 4 mg, preferably in a daily amount ranging from about 4 to about 68 mg, or in an amount of at least about 0.8 mg per 100 ml of liquid, preferably in an amount ranging from about 0.8 to about 14 mg per 100 ml of liquid.

Suitably, the composition A may differ from the composition B and/or the composition C in that the composition A comprises at least one, or at least two, or at least three, or at least four ingredients that is/are not found in the composition B and/or the composition C.

### Composition B

As mentioned before, it is beneficial that the composition B is administrated to a young individual at age from 20 to 30 months as it comprises only specific nutrients that are proved to promote myelination of targeted brain regions, in particular of the social brain, at this specific age following its developmental stage. It therefore avoids the delivery of the nutrients that may have no effect to this process or may even cause negative effect such as sub-optimal myelination leading to impaired or delayed brain development.

Suitably, the composition B is administered to a young individual at age from 20 to 30 months, for example from 21 to 30 months, from 22 to 30 months, from 23 to 30 months, from 24 to 30 months, from 25 to 30 months or from 26 to 30 months.

Composition B is advantageously tailored to be administered to a young individual in myelin-nutrient window 2, to effectively promote myelination in targeted brain region, in particular social brain associated with socio-emotional development, at the inflection period of myelination.

In an embodiment the composition B comprises at least three ingredients chosen from the group consisting of DPA, ARA, sphingomyelin, phosphatidylcholine, octadecatetraenoic acid, oligofructose, 6′-SL, stearidonic acid, beta cryptoxanthin, alpha lactalbumin, gangliosides, phosphatidylinositol, 3′-SL.

In an embodiment the composition B comprises at least three ingredients chosen from the group consisting of DPA, ARA, sphingomyelin, phosphatidylcholine, octadecatetraenoic acid, oligofructose, 6′-SL.

The composition B comprises at least three ingredients chosen from the group consisting of:
DPA in a daily amount of at least about 3 mg, preferably in a daily amount ranging from about 3 mg to about 53 mg, or in an amount of at least about 0.6 mg per 100 ml of liquid, preferably in an amount ranging from about 0.6 to about 11 mg per 100 ml of liquid;
ARA in a daily amount of at least about 0.06 g, preferably in a daily amount ranging from about 0.06 to about 0.4 g, or in an amount of at least about 0.01 g per 100 ml of liquid, preferably in an amount ranging from about 0.01 to about 0.08 g per 100 ml of liquid;
sphingomyelin in a daily amount of at least about 3 mg, preferably in a daily amount ranging from about 3 to about 55 mg, or in an amount of at least about 0.6 mg per 100 ml of liquid, preferably in an amount ranging from about 0.6 to about 11 mg per 100 ml of liquid;
phosphatidylcholine in a daily amount of at least about 0.3 g, preferably in a daily amount ranging from about 0.3 to about 5 g, or in an amount of at least about 0.06 g per 100 ml of liquid, preferably in an amount ranging from about 0.06 to about 1 g per 100 ml of liquid;
octadecatetraenoic acid in a daily amount of at least about 3 mg, preferably in a daily amount ranging from about 3 to about 33 mg, or in an amount of at least about 0.6 mg per 100 ml of liquid, preferably in an amount ranging from about 0.6 to about 7 mg per 100 ml of liquid;
oligofructose in a daily amount of at least about 1 g, preferably in a daily amount ranging from about 1 to about 7 g, or in an amount of at least about 0.2 g per 100 ml of liquid, preferably in an amount ranging from about 0.2 to about 2 g per 100 ml of liquid;
6′-SL in a daily amount of at least about 3 mg, preferably in a daily amount ranging from about 3 to about 23 mg, or in an amount of at least about 0.6 mg per 100 ml of liquid, preferably in an amount ranging from about 0.6 to about 5 mg per 100 ml of liquid;
stearidonic acid in a daily amount of at least about 3 mg, preferably in a daily amount ranging from about 3 to about 41 mg, or in an amount of at least about 0.6 mg per 100 ml of liquid, preferably in an amount ranging from about 0.6 to about 9 mg per 100 ml of liquid;
beta cryptoxanthin in a daily amount of at least about 93 mg, preferably in a daily amount ranging from about 93 to about 484 mg, or in an amount of at least about 19 mg per 100 ml of liquid, preferably in an amount ranging from about 19 to about 98 mg per 100 ml of liquid;
alpha lactalbumin in a daily amount of at least about 0.3 mg, preferably in a daily amount ranging from about 0.3 to about 1 mg, or in an amount of at least about 0.06 mg per 100 ml of liquid, preferably in an amount ranging from about 0.06 to about 0.2 mg per 100 ml of liquid;
gangliosides in a daily amount of at least about 2 mg, preferably in a daily amount ranging from about 2 to about 15 mg, or in an amount of at least about 0.4 mg per 100 ml of liquid, preferably in an amount ranging from about 0.4 to about 3 mg per 100 ml of liquid;
phosphatidylinositol in a daily amount of at least about 4 mg, preferably in a daily amount ranging from about 4 to about 59 mg, or in an amount of at least about 0.8 mg per 100 ml of liquid, preferably in an amount ranging from about 0.8 to about 12 mg per 100 ml of liquid; and
3′-SL in a daily amount of at least about 16 mg, preferably in a daily amount ranging from about 16 to about 113 mg, or in an amount of at least about 3 mg per 100 ml of liquid, preferably in an amount ranging from about 3 to about 23 mg per 100 ml of liquid,
eicosatetraenoic acid in a daily amount of at least about 0.1 g, preferably in a daily amount ranging from about 0.1 to about 0.3 g, or in an amount of at least about 0.06 mg per 100 ml of liquid, preferably in an amount ranging from about 0.01 to about 0.06 mg per 100 ml of liquid.

ARA or DPA is present in natural sources such as for example egg, fungus, algae or fish oil. ARA or DPA may be incorporated in the nutritional compositions of the invention as a single species (as a fatty acid, in the form of a physiologically acceptable salt thereof or in the form of a triglyceride comprising it), as an ingredient consisting of a mixture of different ARA or DPA species or by addition of a natural or synthetic ingredient comprising one or more ARA or DPA species.

Oligofructose can be long chain or short chain, depending on the degree of polymerization of the oligofructose (number of monomers). Preferably the oligofructose of the invention is a short chain oligofructose, most preferably it has a degree of polymerization of from 2 to 10, for example a degree of polymerization of from 2 to 8.

Suitably, the composition B may differ from the composition A and/or the composition C in that the composition B comprises at least one, or at least two, or at least three, or at least four ingredients that is/are not found in the composition A and/or the composition C.

### Composition C

As mentioned before, it is beneficial that the composition C is administrated to a young individual at age from 30 to 66 months, preferably from 30 to 60 months, as it comprises only specific nutrients that are proved to promote myelination of targeted brain regions, in particular of the social brain, at this specific age following its developmental stage. It therefore avoids the delivery of the nutrients that may have no effect to this process or may even cause negative effect such as sub-optimal myelination leading to impaired or delayed brain development.

Suitably, the composition C is administered to a young individual at age from 30 to 66 months, for example from 31 to 66 months, from 32 to 66 months, from 33 to 66 months, from 34 to 66 months, from 35 to 66 months or from 36 to 66 months. In an embodiment, the composition C is administered to a young individual at age from 30 to 60 months, for example from 31 to 60 months, from 32 to 60 months, from 33 to 60 months, from 34 to 60 months, from 35 to 60 months or from 36 to 60 months.

Composition C is advantageously tailored to be administered to a young individual in myelin-nutrient window 3, to effectively promote myelination in targeted brain region, in particular social brain associated with socio-emotional development, at the stage where a slower increase of myelination is observed.

In a preferred embodiment of the invention the composition C comprises at least three ingredients chosen from the group consisting of DHA, sphingomyelin, phosphatidylcholine, histidine, isoleucine, leucine, valine, alpha carotene, folic acid, alpha lactalbumin, gangliosides, phosphatidylinositol, phosphatidylethanolamine, eicosenoic acid, stearidonic acid, DPA, 3′-SL, 6′-SL, alanine, arginine, aspartic acid, cystine, glutamic acid, glycine, lysine, methionine, phenylalanine, proline, serine, threonine, tryptophan, tyrosine, alpha tocopherol, octadecatetraenoic acid, eicosapentaenoic acid, phosphorus, phosphatidylserine.

In an embodiment of the invention the composition C comprises at least three ingredients chosen from the group consisting of DHA, sphingomyelin, phosphatidylcholine, histidine, isoleucine, leucine, valine, alpha carotene, folic acid, alpha lactalbumin, gangliosides, phosphatidylinositol, phosphatidylethanolamine, eicosenoic acid, stearidonic acid, DPA, 3′-SL, 6′-SL, alanine, arginine, aspartic acid, cystine, glutamic acid, glycine, lysine, methionine, phenylalanine, proline, serine, threonine, tryptophan, tyrosine.

In a preferred embodiment the composition C comprises at least three ingredients chosen from the group consisting of DHA, sphingomyelin, phosphatidylcholine, histidine, isoleucine, leucine, valine.

The composition C comprises at least three ingredients chosen from the group consisting of:
DHA in a daily amount of at least about 0.01 g, preferably in a daily amount ranging from about 0.01 to about 0.3 g, or in an amount of at least about 0.01 g per 100 ml of liquid, preferably in an amount ranging from about 0.01 to about 0.06 g per 100 ml of liquid;
sphingomyelin in a daily amount of at least about 5 mg, preferably in a daily amount ranging from about 5 to about 96 mg, or in an amount of at least about 1 mg per 100 ml of liquid, preferably in an amount ranging from about 1 to about 20 mg per 100 ml of liquid;
phosphatidylcholine in a daily amount of at least about 0.4 g, preferably in a daily amount ranging from about 0.4 to about 4 g, or in an amount of at least about 0.08 g per 100 ml of liquid, preferably in an amount ranging from about 0.08 to about 0.8 g per 100 ml of liquid;
histidine in a daily amount of at least about 0.3 g, preferably in a daily amount ranging from about 0.3 to about 1 g, or in an amount of at least about 0.06 g per 100 ml of liquid, preferably in an amount ranging from about 0.06 to about 0.2 g per 100 ml of liquid;
isoleucine in a daily amount of at least about 0.5 g, preferably in a daily amount ranging from about 0.5 to about 2 g, or in an amount of at least about 0.1 g per 100 ml of liquid, preferably in an amount ranging from about 0.1 to about 0.4 g per 100 ml of liquid;
leucine in a daily amount of at least about 1 g, preferably in a daily amount ranging from about 1 to about 4 g, or in an amount of at least about 0.2 g per 100 ml of liquid, preferably in an amount ranging from about 0.2 to about 0.8 g per 100 ml of liquid;
valine in a daily amount of at least about 0.7 g, preferably in a daily amount ranging from about 0.7 to about 3 g, or in an amount of at least about 0.1 g per 100 ml of liquid, preferably in an amount ranging from about 0.1 to about 0.6 g per 100 ml of liquid;
alpha carotene in a daily amount of at least about 214 mg, preferably in a daily amount ranging from about 214 to about 2087 mg, or in an amount of at least about 43 mg per 100 ml of liquid, preferably in an amount ranging from about 43 to about 424 mg per 100 ml of liquid;
folate in daily amount of at least about 251 μg, preferably in daily amount ranging from about 251 to about 400 μg, or in an amount of at least about 51 μg per 100 ml of liquid, preferably in an amount ranging from about 51 to about 82 μg per 100 ml of liquid;
alpha lactalbumin in a daily amount of at least about 0.1 mg, preferably in a daily amount ranging from about 0.1 to about 1 mg, or in an amount of at least about 0.02 mg per 100 ml of liquid, preferably in an amount ranging from about 0.02 to about 0.2 mg per 100 ml of liquid;
gangliosides in a daily amount of at least about 2 mg, preferably in a daily amount ranging from about 2 to about 16 mg, or in an amount of at least about 0.4 mg per 100 ml of liquid, preferably in an amount ranging from about 0.4 to about 3 mg per 100 ml of liquid;
phosphatidylinositol in a daily amount of at least about 0.01 g, preferably in a daily amount ranging from about 0.01 to about 0.2 g, or in an amount of at least about 0.01 g per 100 ml of liquid, preferably in an amount ranging from about 0.01 to about 0.04 g per 100 ml of liquid;
phosphatidylethnolamine in a daily amount of at least about 0.05 g, preferably in a daily amount ranging from about 0.05 to about 0.9 g, or in an amount of at least about 0.01 g per 100 ml of liquid, preferably in an amount ranging from about 0.01 to about 0.2 g per 100 ml of liquid;
eicosenoic acid in a daily amount of at least about 0.1 g, preferably in a daily amount ranging from about 0.1 to about 0.36 g, or in an amount of at least about 0.02 g per 100 ml of liquid, preferably in an amount ranging from about 0.02 to about 0.1 g per 100 ml of liquid;
stearidonic acid in daily amount of at least about 3 mg, preferably in daily amount ranging from about 3 to about 70 mg, or in an amount of at least about 0.6 mg per 100 ml of liquid, preferably in an amount ranging from about 0.6 to about 14 mg per 100 ml of liquid;
DPA in a daily amount of at least about 8 mg, preferably in a daily amount ranging from about 8 to about 46 mg, or in an amount of at least about 2 mg per 100 ml of liquid, preferably in an amount ranging from about 2 to about 9 mg per 100 ml of liquid;
3′-SL in a daily amount of at least about 6 mg, preferably in a daily amount ranging from 6 to about 102 mg, or in an amount of at least about 1 mg per 100 ml of liquid, preferably in an amount ranging from about 1 to about 21 mg per 100 ml of liquid;
6′SL in a daily amount of at least about 2 mg, preferably in a daily amount ranging from about 2 to about 21 mg, or in an amount of at least about 0.4 mg per 100 ml of liquid, preferably in an amount ranging from about 0.4 to about 4 mg per 100 ml of liquid;
alanine in a daily amount of at least about 0.5 g, preferably in a daily amount ranging from about 0.5 to about 2 g, or in an amount of at least about 0.1 g per 100 ml of liquid, preferably in an amount ranging from about 0.1 to about 0.4 g per 100 ml of liquid;
arginine in a daily amount of at least about 0.5 g, preferably in a daily amount ranging from about 0.5 to about 2 g, or in an amount of at least about 0.1 g per 100 ml of liquid, preferably in an amount ranging from about 0.1 to about 0.4 g per 100 ml of liquid;
aspartic acid in a daily of at least about 1 g, preferably in a daily amount ranging from about 1 to about 4 g, or in an amount of at least about 0.2 g per 100 ml of liquid, preferably in an amount ranging from about 0.2 to about 0.8 g per 100 ml of liquid;
cystine in a daily amount of at least about 0.1 g, preferably in a daily amount ranging from about 0.1 to about 0.6 g, or in an amount of at least about 0.02 g per 100 ml of liquid, preferably in an amount ranging from about 0.02 to about 0.1 g per 100 ml of liquid;
glutamic acid in a daily of at least about 2 g, preferably in a daily amount ranging from about 2 to about 11 g, or in an amount of at least about 0.4 g per 100 ml of liquid, preferably in an amount ranging from about 0.4 to about 2 g per 100 ml of liquid;
glycine in a daily amount of at least about 0.3 g, preferably in a daily amount ranging from about 0.3 to about 2 g, or in an amount of at least about 0.06 g per 100 ml of liquid, preferably in an amount ranging from about 0.06 to about 0.4 g per 100 ml of liquid;
lysine in a daily amount of at least about 0.7 g, preferably in a daily amount ranging from about 0.7 to about 3 g, or in an amount of at least about 0.1 g per 100 ml of liquid, preferably in an amount ranging from about 0.1 to about 0.6 g per 100 ml of liquid;
methionine in a daily amount of at least about 0.2 g, preferably in a daily amount ranging from about 0.2 to about 1 g, or in an amount of at least about 0.04 g per 100 ml of liquid, preferably in an amount ranging from about 0.04 to about 0.2 g per 100 ml of liquid;
phenylalanine in a daily amount of at least about 0.6 g, preferably in a daily amount ranging from about 0.6 to about 2 g, or in an amount of at least about 0.1 g per 100 ml of liquid, preferably in an amount ranging from about 0.1 to about 0.4 g per 100 ml of liquid;
proline in a daily amount of at least about 1 g, preferably in a daily amount ranging from about 1 to about 4 g, or in an amount of at least about 0.2 g per 100 ml of liquid, preferably in an amount ranging from about 0.2 to about 0.8 g per 100 ml of liquid;
serine in a daily amount of at least about 0.6 g, preferably in a daily amount ranging from about 0.6 to about 2 g, or in an amount of at least about 0.1 g per 100 ml of liquid, preferably in an amount ranging from about 0.1 to about 0.6 g per 100 ml of liquid;
threonine in a daily amount of at least about 0.5 g, preferably in a daily amount ranging from about 0.5 to about 2 g, or in an amount of at least about 0.1 g per 100 ml of liquid, preferably in an amount ranging from about 0.1 to about 0.4 g per 100 ml of liquid;
tryptophan in a daily amount of at least about 0.1 g, preferably in a daily amount ranging from about 0.1 to about 0.8 g, or in an amount of at least about 0.02 g per 100 ml of liquid, preferably in an amount ranging from about 0.02 to about 0.2 g per 100 ml of liquid;
tyrosine in a daily amount of at least about 0.5 g, preferably in a daily amount ranging from about 0.5 to about 2 g, or in an amount of at least about 0.1 g per 100 ml of liquid, preferably in an amount ranging from about 0.1 to about 0.4 g per 100 ml of liquid;
alpha tocopherol in a daily amount of at least about 6 mg, preferably in a daily amount ranging from about 6 to about 18 mg, or in an amount of at least about 1.2 mg per 100 ml of liquid, preferably in an amount ranging from about 1.2 to about 3.7 mg per 100 ml of liquid;
octadecatetraenoic acid in a daily amount of at least about 6.8 mg, preferably in a daily amount ranging from about 6.8 to about 56 mg, or in an amount of at least about 1.3 mg per 100 ml of liquid, preferably in an amount ranging from about 1.3 to about 11.5 mg per 100 ml of liquid;
eicosapentaenoic acid in a daily amount of at least about 8 mg, preferably in a daily amount ranging from about 8 to about 127 mg, or in an amount of at least about 1.6 mg per 100 ml of liquid, preferably in an amount ranging from about 1.6 to about 26 mg per 100 ml of liquid;
phosphorus in a daily amount of at least about 894 mg, preferably in a daily amount ranging from about 894 to about 1475 mg, or in an amount of at least about 183 mg per 100 ml of liquid, preferably in an amount ranging from about 183 to about 301 mg per 100 ml of liquid;
phosphatidylserine in a daily amount of at least about 18 mg, preferably in a daily amount ranging from about 18 to about 294 mg, or in an amount of at least about 0.1 mg per 100 ml of liquid, preferably in an amount ranging from about 0.1 to about 60 mg per 100 ml of liquid.

DHA is present in natural sources such as for example egg, algae or fish oil. According to an embodiment, DHA is provided in the form of triglycerides comprising DHA.

Oils comprising DHA and generally other polyunsaturated fatty acids (PUFAs) may be of various origin. Preferably, the DHA is provided in the form of a fish oil comprising DHA. Fish oils generally comprise 5 wt. % or more, preferably 10 wt. % or more of DHA. Oils comprising substantial amounts of DHA obtained from algae or microorganisms in general are also available. For example, oils harvested from algae comprising 10 wt. % or more, for example 20 wt. % or more of DHA may be used.

DHA may be incorporated in the nutritional compositions of the invention as a single species (as a fatty acid, in the form of a physiologically acceptable salt thereof or in the form of a triglyceride comprising it), as an ingredient consisting of a mixture of different DHA species or by addition of a natural or synthetic ingredient comprising one or more DHA species.

Suitably, the composition C may differ from the composition A and/or the composition B in that the composition C comprises at least one, or at least two, or at least three, or at least four ingredients that is/are not found in the composition A and/or the composition B.

In one embodiment, the present invention relates to the kit as described above wherein each of said compositions comprise at least four said ingredients chosen from said group.

In one embodiment, the present invention relates to the kit as described above wherein each of said compositions comprise at least five said ingredients chosen from said group.

In one embodiment, the present invention relates to the kit as described above wherein each of said compositions comprise at least six said ingredients chosen from said group.

Composition A, composition B or composition C of the kit of the invention may be any type of composition suitable for direct administration to a subject.

In particular a composition of the kit may be a synthetic nutritional composition.

The person skilled in the art would identify appropriate amounts of the above-mentioned nutrients, metabolic precursors or metabolites thereof to achieve in the nutritional composition after administration their highest permitted levels.

### Preferred Nutritional Composition Matrix

Composition A, composition B or composition C according to the invention can be a synthetic nutritional composition. It can be liquids, powders, gels, pastes, solids, concentrates, suspensions, or ready-to-use forms of enteral formulas, oral formulas, formulas for infants, formulas for pediatric subjects, formulas for children, growing-up milks and/or formulas, baby food.

In one typical embodiment of the present invention, the composition will contain a protein source, a lipid source and a carbohydrate source.

For example, such a composition may comprise protein in the range of about 2 to 6 g/100 kcal, lipids in the range of about 1.5 to 3 g/100 kcal and/or carbohydrates in the range of about 1.7 to 12 g/100 kcal.

If the composition is liquid, its energy density may be between 60 and 75 kcal/100 ml. If the composition is solid, its energy density may be between 60 and 75 kcal/100 g.

The type of protein is not believed to be critical to the present invention. Thus, protein sources based on whey, casein and mixtures thereof may be used, for example. As far as whey proteins are concerned, acid whey or sweet whey or mixtures thereof may be used as well.

The proteins may be intact or hydrolysed or a mixture of intact and hydrolysed proteins. It may be desirable to supply partially hydrolysed proteins (degree of hydrolysis between 2 and 20%), for example for subjects believed to be at risk of developing cows' milk allergy. If hydrolysed proteins are required, the hydrolysis process may be carried out as desired and as is known in the art. For example, a whey protein hydrolysate may be prepared by enzymatically hydrolysing the whey fraction in two steps as described in EP 322589. For an extensively hydrolysed protein, the whey proteins may be subjected to triple hydrolysis using Alcalase 2.4 L (EC 940459), then Neutrase 0.5 L (obtainable from Novo Nordisk Ferment AG) and then pancreatin at 55° C. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

The compositions of the present invention may contain a carbohydrate source. Any carbohydrate source may be used, such as lactose, saccharose, maltodextrin, starch, honey and mixtures thereof.

The compositions of the present invention may contain a lipid source. The lipid source may be any lipid. Preferred fat sources include milkfat and vegetable oils (including but not limited to palm oil, high oleic sunflower oil and high oleic safflower oil). The essential fatty acids linoleic and a-linolenic acid may also be added.

Apart from the above-mentioned, the compositions may optionally contain other substances which may have a beneficial effect such as probiotics, prebiotics, nucleotides, nucleosides, and the like.

The compositions, for example an infant formula, for use in the invention may be prepared in any suitable manner. For example, an infant formula may be prepared by blending together the protein source, the carbohydrate source, and the fat source in appropriate proportions. If used, emulsifiers may be included in the blend. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The liquid mixture may then be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger, for example, a plate heat exchanger. The liquid mixture may then be cooled to about 60° C. to about 85° C.; for example, by flash cooling. The liquid mixture may then be homogenized; for example, in two stages at about 7 MPa to about 40 MPa in the first stage and about 2 MPa to about 14 MPa in the second stage. The homogenized mixture may then be further cooled to add any heat sensitive components, such as vitamins and minerals. The pH and solids content of the homogenized mixture is conveniently standardized at this point. The homogenized mixture is transferred to a suitable drying apparatus such as a spray drier or freeze drier and converted to powder. The powder should have a moisture content of less than about 5% by weight. If it is desired to add probiotic(s), they may be cultured according to any suitable method and prepared for addition to the infant formula by freeze-drying or spray-drying for example. Alternatively, bacterial preparations can be bought from specialist suppliers such as Christian Hansen and Morinaga already prepared in a suitable form for addition to food products such as infant formula. Such bacterial preparations may be added to the powdered infant formula by dry mixing.

A composition of the kit, for example, may be in a solid form. A composition may, for example, be in the form of a chewable tablet, dispersible tablet, capsule, lozenge, pastille, chewing gum, powder (e.g. in a sachet), stickpack sachets, or bottle with powder in the cap. The tablet or capsule may be provided as a unit dosage form. A powder composition may be contained in a sachet. A powder composition according to the present invention may be used to sprinkle onto a food or beverage. A particularly preferred embodiment provides a composition according to the invention in the form of a sachet containing a powder, wherein the powder can be dispersed into a beverage (e.g. water, fruit juice, milk, etc.) to provide a palatable nutrient liquid for oral administration.

As an example, it may be a dairy product, a chilled or shelf stable beverage or a soup, a dietary supplement, a meal replacement, or a nutritional bar.

### Use of the Kit

To promote age-dependent brain development, more specifically myelination, of targeted brain regions, more specific of the social brain regions associated with social-emotional skills, performance and development, such as understanding and interacting with others, experiencing, managing, and expressing a full range of positive and negative emotions, developing close, satisfying relationships with others, including emotional skills, social skills, sociability, social aspects of executive functions, happiness, positive mood, social cognition, social communication, social decision-making, self-regulation, compliance, adaptive functioning, affect, social communication, interaction, autonomy, each one of the compositions of the kit should be delivered to a child at the appropriate age window, as defined by the inventors and described above, that is sensitive for the intake of the age and brain stage specific ingredients comprised in said composition.

Sequential administration of the compositions of the kit follows a staged feeding regimen of a young individual as claimed, comprising the steps of:
a) administering the composition A as described above at age from 0 to 20 months, preferably from 6 to 20 months;
b) administering the composition B as described above at age from 20 to 30 months;
c) administering the composition C as described above at age from 30 to 66 months, preferably from 30 to 60 months.

In one embodiment of the invention composition A and composition B of the kit are administered at corresponding age windows.

In one embodiment of the invention composition A and composition C of the kit are administered at corresponding age windows.

In one embodiment of the invention composition B and composition C of the kit are administered at corresponding age windows.

In one embodiment of the invention composition A and composition B and composition C of the kit are administered at corresponding age windows.

### Health Effect

As it was found by the inventors, the factors/nutrients having impact on myelination of specific brain regions are different at different age stages of a child's development and the administration of some of the nutrients that are known from prior art to promote overall brain myelination during “non-sensitive” stage may have no effect or even have negative effect on this process, in particular in specific brain regions, such as social brain, which has not been considered in prior art.

The nutrients content in each age-tailored composition of the kit of the invention is of particular importance at various age of the brain development of a child. Each nutrient can be related to promoting myelination of a targeted brain region at specific age, and thus impacts brain development. Each nutrient (presence and amount content) may be correctly balanced with other nutrients of the compositions to ensure the proper age-staged brain development, more specifically myelination, of targeted brain regions such as social brain.

The kit of the invention has a positive effect on the myelination in the brain of young individuals, in particular in children who are provided with the compositions of the kit at corresponding sensitive age window.

Such positive effect can comprise brain development, more specifically myelination, of targeted brain regions associated with social-emotional skills, performance and development of children, such as understanding and interacting with others, experiencing, managing, and expressing a full range of positive and negative emotions, developing close, satisfying relationships with others, including emotional skills, social skills, sociability, social aspects of executive functions, happiness, positive mood, social cognition, social communication, social decision-making, self-regulation, compliance, adaptive functioning, affect, social communication, interaction, autonomy.

It may also prevent impaired or delayed development.

The health effect can be observed after a few days, weeks or months of use of the kit.

The effect of the invention can be preventive (for example avoiding a sub-optimal myelination in targeted brain regions) or curative (restoring an optimal myelination of targeted brain regions).

The health effect related to children can be measured by various methods as illustrated in the example below.

To summarize, by modifying the contents of nutrients in the compositions according to a child's age, the kit of nutrient blend compositions of the present invention can provide optimum nutrient contents which follow the nutritional supply demands of the age-staged myelination of brain structure, thus it is beneficial for the brain development, more specifically myelination, of targeted brain regions associated with social-emotional skills, performance and development of children at specific ages.

### Examples

The inventors have investigated the developmental dynamics of nutrient-myelin interactions from infancy to early childhood, using myelin imaging as a marker for brain maturation. Brain neuroimaging and infant nutritional intake information were analyzed from 293 children from a longitudinal cohort study in the US. A sliding window approach was used to investigate the relationship between the nutrient intake and brain region myelination over a continuous set of age windows.

### Material and Methods

### Participants

Participants for this study are part of a larger longitudinal pre- and post-natal study of child health and development in the USA. The study aims to understand the relationships between child genomics, nutrition, and home environmental exposures on brain and cognitive development. Infants and young children were recruited between the ages of birth and 5 years of age with study visits scheduled biannually from birth through 30 months of age, and annually thereafter.

For inclusion in the analysis of the present study, 293 children between 6 months and 60 months of age were selected who met the following criteria: 1. Singleton full-term (>37 weeks) pregnancy; 2. Uncomplicated pregnancy (i.e., no reports of pre-eclampsia, gestational hypertension or gestation diabetes mellitus, GDM); 3. No reports of abnormalities on fetal ultrasound; 4. Infant 5 minute APGAR scores ≥8; 5. Infant birth weight greater than 1500 g; 6. No reported history of neurological trauma in the child; 7. No reported sibling or parental psychiatric history, including autism; and 8. No reports of major depressive disorder requiring medication in the mother during pregnancy or in the 6 months prior to becoming pregnant. In addition, children had to have a successful MRI scan and time-matched nutrition intake information for at least one 24-hour period within a week of the MRI. Demographic information is summarized in Table 1.

TABLE 1
Demographic information for study population

| Biological Sex (N) | Male | 165 | |
|---|---|---|---|
| | Female | 128 | |
| Age (months) (Mean (SD) | | 25.5 | (4.5) |
| Birth Weight (lbs) | | 7.4 | (1.2) |
| Birth Length (inches) | | 20.2 | (1.9) |
| Maternal Education (N) | Professional Degree | 105 | |
| | College Graduate | 74 | |
| | Partial College | 49 | |
| | High School Graduate | 21 | |
| | Partial High School | 3 | |
| | Not Reported | 41 | |
| Race (N) | White | 181 | |
| | Black or African American | 36 | |
| | Asian | 13 | |
| | Mixed | 22 | |
| | Not Reported | 41 | |
| Cognitive Development | MSEL ELC | 101 | (18) |
| Composite (Mean (SD)) | MSEL VDQ | 99 | (22) |
| | MSEL NVDQ | 105 | (18) |

### Myelin MRI Data Acquisition and Processing

All MR imaging was performed at 3 Tesla (Siemens Tim Trio) during natural and non-sedated sleep as described in Dean DC. et al. Pediatr. Radiol. 2014; 44 (1): 64-72.

To measure white matter development and myelination, mcDESPOT multicomponent T₁ and T₂ relaxometry data were collected using age-optimized protocols (Table 2). A consistent voxel dimension of ^{˜}(1.8×1.8×1.8) mm³ was used across the age span, with the field of view and imaging matrix adjusted depending on child age and head size. The theoretical basis for DESPOT1 and mcDESPOT has been detailed previously Dean DC. et al. Pediatr. Radiol. (2014); 44 (1): 64-72; Deoni S C. et. al. Magn. Reason. Med. 2008; 60 (6): 1372-87) and involves the acquisition of multi-flip angle T₁-weighted SPGR images (DESPOT1), as well as T₁/T₂-weighted bSSFP images at two differing radiofrequency (RF) phase-cycling patterns. From these data, single and multi-component relaxometry measures are calculated. For multicomponent analysis, a 3-pool tissue model comprising myelin-associated, intra and extra cellular, and CSF water pools is fit to the combined SPGR and bSSFP data using an iterative stochastic region approach (38).

TABLE 2
Age-optimized imaging protocols for qT₁, qT₂, and MWF imaging.

| | | 3-9 Months | 9-16 Months | 16-28 Months | 28-48 Months | >48 Months |
|---|---|---|---|---|---|---|
| | Field of | 14 × 14 × 13 | 17 × 17 × 14.4 | 18 × 18 × 15 | 20 × 20 × 15 | 20 × 20 × 15 |
| | View (cm) | | | | | |
| SPGR | TE/TR (ms) | 5.8/12 | 5.9/12 | 5.4/12 | 5.2/11 | 4.2/8 |
| | Flip Angles | 2, 3, 4, 5, 7, | 2, 3, 4, 5, 7, | 2, 3, 4, 5, 7, | 2, 3, 4, 5, 7, | 3, 4, 5, 6, 7, |
| | (degrees) | 9, 11, 14 | 9, 11, 14 | 9, 11, 14, | 9, 12, 16 | 9, 13, 18 |
| | Bandwidth | 350 | | | | |
| | (Hz/Pixel) | | | | | |
| | Image Matrix | 80 × 80 × 72 | 96 × 96 × 80 | 104 × 104 × 84 | 112 × 112 × 84 | 112 × 112 × 84 |
| IR-SPGR | TI/TE/TR (ms) | (600, 950)/5.8/12 | (600, 900)/5.9/12 | (500, 850)/5.4/12 | (500, 800)/5.2/11 | (450, 750)/4.8/10 |
| | Flip Angle | 5 | | | | |
| | (degrees) | | | | | |
| | Image Matrix | 80 × 80 × 36 | 96 × 96 × 40 | 108 × 104 × 42 | 112 × 112 × 42 | 112 × 112 × 42 |
| bSSFP | TE/TR (ms) | 5/10 | 5.1/10.2 | 5/10 | 4.4/9.8 | 3.6/7.2 |
| | Flip Angles | 9, 14, 20, 27, 34, 41, 56, 70 | | | | |
| | (degrees) | | | | | |
| | Bandwidth | 350 | | | | |
| | (Hz/Pixel) | | | | | |
| | Image Matrix | 80 × 80 × 72 | 96 × 96 × 80 | 104 × 104 × 84 | 112 × 112 × 84 | 112 × 112 × 84 |
| | Field of | 20 × 20 × 15 | | | | |
| | View (cm) | | | | | |
| High | TI/TE/ | 950 ms/6.9 ms/16 ms | | | | |
| Resolution | TR (ms) | | | | | |
| IR-SPGR | Flip Angle | 5 | | | | |
| | (degrees) | | | | | |
| | Reconstructed | 224 × 224 × 150 | | | | |
| | Image Matrix | | | | | |

From the acquired mcDESPOT data, the myelin water fraction (MWF) was calculated using a processing pipeline that includes linear alignment of the SPGR, IR-SPGR, and bSSFP images to account for subtle intra-scan head movement, non-parenchyma voxel removal, and correction of flip angle errors and off-resonance inhomogeneities using DESPOT1-HIFI and DESPOT2-FM. A 3-pool tissue model was fit to the multi-angle SPGR and bSSFP data to estimate the MWF, a surrogate and non-invasive measure of myelin content. The MWF images (maps) were then non-linearly aligned to a common analysis space in approximate MNI space using a previously described multi-step approach that first aligns the subject's high flip angle T1 weighted SPGR image to one of 14 age-specific templates and then to an overall study-specific template using non-linear 3D deformation, ANTS. The calculated transformation matrices are then applied to the quantitative MWF maps.

### Nutritional Data Collection

Nutrient intake data was collected through the Automated Self-Administered 24-hour dietary assessment tool (ASA-24). The average daily nutrient intake per scan age was calculated, resulting in 293 observations with 88 nutrients. Values for amino acids and phospholipids, including sphingomyelin, were retrieved from the relevant United States Department of Agriculture (USDA) databases. The USDA choline database contains the values of free choline, glycerophosphocholine, phosphocholine, phosphatidylcholine and sphingomyelin for about 630 foods. Ganglioside values for meat and fish were estimated from Khor et al. BMC Nutrition 2016; 2(1): 74 and for milk the inventors assumed 11 mg of ganglioside per liter as suggested by Vesper et al. Journal of Nutrition 1999; 129(7):1239-50. The oligosaccharides 3′SL and 6′SL are only found in milk products, values of 114 and 23 mg/100 g were used for their respective concentrations in cow's milk. For the estimation of oligofructose, the inventors based their calculations on Moshfegh et al. J. Nutr. 1999; 129(7 Suppl): 1407s-11s. We then mapped the foods reported in ASA-24 to the corresponding items in the USDA database and calculated the daily totals.

### Brain-Nutrient Associations Across Windows

With the MWF maps in standard space, regional mean values were calculated across the brain in each child. The combined data from all 293 children was used to identify dynamics and window size and to perform a series of MWF-nutrient correlations using a sliding-window approach. For each nutrient of interest and for each brain region, the inventors set a window size of 25 myelin imaging measurements corresponding to a step size of 72 to 162 days and fit the following general linear model to the data:

$${MWF}_{i , j} = {β}_{0 , j} + {β}_{1} log ( {age}_{i , j} ) + {β}_{2} {NutrientIntake}_{i , j} + {β}_{3} {NutrientIntake}_{i , j} \times log ( {age}_{i , j} ) . \qquad [ 1 ]$$

In the model of the inventors, MWFi,j is the mean regional myelin water fraction estimate in child j at timepoint i, age_{i,j} is the corresponding child age at the timepoint, and β₀, β₁, β₂, and β₃ are the regression coefficients. Once the general linear model was fit for all age-nutrient-brain myelin combinations, the inventors then plotted the resultant p value ‘map’ (unadjusted p<0.05) associated with the β₂ nutrient intake term, with the age-wise p values along the x axis, and nutrients along y. The inventors focused on the direct nutrient intake term since the sliding window approach provided information on the relationship with age.

Finally, the inventors used a sobel-filter vertical edge-detection with a threshold of 25 to identify age windows with differing patterns of nutrient associations. Nutrients per window were selected based on a threshold of 50% of significance for positive associations in each window (i.e., within the age window they had an unadjusted p-value<0.05 for at least half the age points).

### Nutrient Intake Dynamics Per Nutrient-Myelin Window

The samples were split after window identification according to the following age windows: [6, 20) months, [20, 30) months, and [30, 60) months. For each nutrient, and within each window, a linear model was fit to the data, with the nutrient intake as response and the scan age as independent variable. An increasing trend was defined as a positive and a decreasing trend as a negative slope in the linear model, with a p-value below 0.05. A non-significant slope was considered as a ‘stable trend’. In addition, a linear model was fit to the data for all ages, for each nutrient.

Association of Myelin with Nutrient Intakes Across Windows

A linear model, like [1], was fitted to the dataset, all ages included, for each nutrient. Linear models with significant and positive coefficients for the nutrient intake terms were considered as indicative of a linear positive correlation with myelin. The myelin water fraction was Box Cox transformed to make it more symmetric. In addition, to account for possibly non-linear associations and for correlations between the nutrients, we performed a regression model using a random forest and used a feature selection algorithm for finding all relevant variables.

### Results

### Brain-Nutrient Associations (Nutrient-Myelin Windows)

The majority of nutrients assessed (60%) were positively associated with brain maturation over the early developmental period from 6 to 20 months of age (window 1); then a period of relative stability, during which only one-fifth of nutrients (20%) had a significant association with brain myelination (20 to 30 months, window 2); and finally, a refractive period of growth during which the 37% of nutrients were positively associated with brain development (30 to 60 months, window 3). The inventors note that in all cases, the associations were positive, i.e., higher nutrient intake associated with higher measures of brain myelin. This likely reflects the study sample, which are healthy, neuro-typically developing children living in a high resource area of the United States. Lastly, the inventors observed differences in nutrient-myelin dynamics across age windows, for example amino acids were positively associated with myelin at later ages only (windows 2 and 3), unlike minerals (window 1) or vitamins (windows 1 and 3, but not window 2), see Table 3.

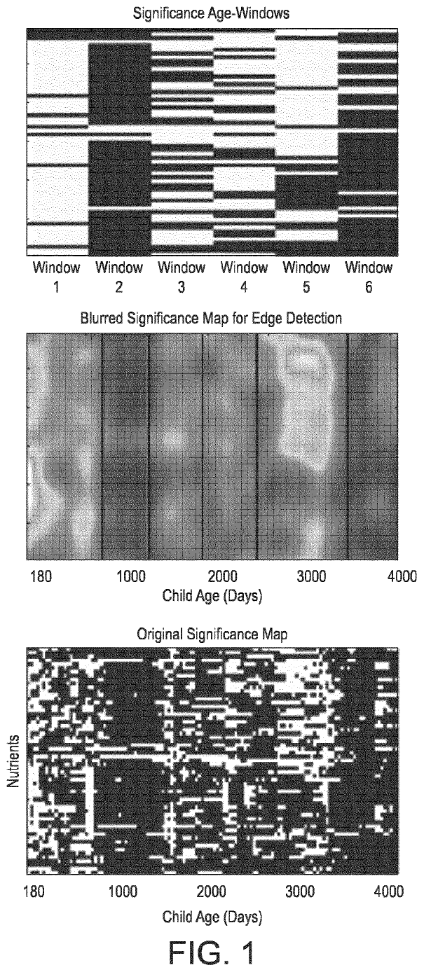

FIG. 1 displays the nutrient versus age p-value map calculated from the sliding window analysis and summarized into the main age windows. The analysis was initially done on the full sample up to 12 years of age until the windows were identified that cover the target age range from 1 to 5 years.

TABLE 3
Nutrients with significant associations to myelin per window

| | Window 1 | Window 2 | Window 3 |
|---|---|---|---|
| | (N = 56) | (N = 20) | (N = 35) |
| Macronutrients | Carbohydrate, Protein, | Carbohydrate | Carbohydrate, Protein |
| | Dietary fiber, Total fat | | |
| Micronutrients | Vitamin E as α- | / | Retinol, Vitamin C, |
| (vitamins) | tocopherol, Folic acid², | | Choline |
| | Folate³, Food folate⁴, | | |
| | Niacin, Retinol, β- | | |
| | cryptoxanthin, Vitamin A, | | |
| | Thiamine, Vitamin B-12, | | |
| | riboflavin, Vitamin B-6, | | |
| | Vitamin C, Vitamin D, | | |
| | Vitamin K, Choline | | |
| Micronutrients | Calcium, Iron, | / | / |
| (minerals) | Magnesium, Phosphorus, | | |
| | Potassium | | |
| | Selenium, Zinc | | |
| Other compounds | Gangliosides, | Sphingomyelin | Gangliosides, |
| | Oligofructose, | | Oligofructose, |
| | Sphingomyelin | | Sphingomyelin |
| | Lycopene | | |
| | Theobromine | | |
| | Cholesterol | | |
| Fatty acids | Hexadecenoic | Eicosatetraenoic (ETA, | Hexadecenoic |
| | (Palmitoleic, 16:1), | 20:4), Docosapentaenoic | (Palmitoleic, 16:1), |
| | Octadecenoic (Oleic, | (DPA, 22:5), | Eicosenoic (20:1), |
| | 18:1), Docosenoic (Erucic, | | Octadecatetraenoic |
| | 22:1), Total | | (18:4), Eicosatetraenoic |
| | monounsaturated fatty | | (ETA, 20:4), |
| | acids, Octadecadienoic | | Eicosapentaenoic (EPA, |
| | (Linoleic, 18:2), | | 20:5), Docosapentaenoic |
| | Octadecatrienoic (α- | | (DPA, 22:5), |
| | linolenic acid, 18:3), | | Docosahexaenoic (DHA, |
| | Eicosatetraenoic (ETA, | | 22:6), Total |
| | 20:4), Docosapentaenoic | | polyunsaturated fatty |
| | (DPA, 22:5), | | acids, |
| | Docosahexaenoic (DHA, | | |
| | 22:6), Total | | |
| | polyunsaturated fatty | | |
| | acids, Butanoic acid (4:0), | | |
| | Hexanoic acid (6:0), | | |
| | Octanoic acid (8:0), | | |
| | Decanoic acid (10:0), | | |
| | Dodecanoic acid (12:0), | | |
| | Tetradecanoic (14:0), | | |
| | Hexadecanoic (16:0), | | |
| | Octadecanoic (18:0), | | |
| | Total saturated fatty | | |
| | acids, | | |
| Phospholipids | Phosphatidylcholine | Phosphatidylcholine | Phosphatidylcholine |
| | Phosphatidylinositol | Phosphatidylinositol | Phosphatidylinositol |
| | Phosphatidylserine | | Phosphatidylserine |
| | Phosphatidyethanolamine | | Phosphatidyethanolamine |
| Amino acids | / | Alanine, Aspartic acid, | Alanine, Aspartic acid, |
| | | Cysteine, Glutamic acid, | Cysteine, Glutamic acid, |
| | | Glycine, Isoleucine, | Glycine, Isoleucine, |
| | | Leucine, Lysine, | Leucine, Lysine, |
| | | Methionine, | Methionine, |
| | | Phenylalanine, Proline, | Phenylalanine, Proline, |
| | | Serine, Tyrosine, Valine | Serine, Threonine, |
| | | | Tyrosine, Valine |

¹ Age window 1 from 6-20 months, window 2 from 20-30 months and window 3 from 30 to 60 months.
²Folic acid is the fully oxidized monoglutamate form of the vitamin that is used in fortified foods and most dietary supplements.
³Folate is the generic term for naturally occurring food folates, and folates in dietary supplements and fortified foods, including folic acid.
⁴Food folates naturally occur in food and are in the tetrahydrofolate (THF) form and usually have additional glutamate residues, making them polyglutamates.

### Nutrient Intake Dynamics for Nutrients Per Window

The nutrient intake ranges and medians are summarized in Table 4.

TABLE 4
Daily nutrient intakes mean and minimum (min) to maximum
(max) ranges for nutrients per age window^{a}.

| | | |
|---|---|---|
| Nutrient | Mean (SD) | Min-Max |

Age window 6-20 months

| | | | |
|---|---|---|---|
| Copper (mg) | 0.6 | (0.2) | 0.2-1.1 |
| Folate (mcg) | 179.9 | (85.3) | 29.3-459.1 |
| Histidine (g) | 0.2 | (0.1) | 0-0.7 |
| Iron (mg) | 9 | (5.2) | 0.6-23 |
| Isoleucine (g) | 0.4 | (0.3) | 0-1.2 |
| Leucine(g) | 0.8 | (0.6) | 0-3.1 |
| Magnesium (mg) | 144.6 | (54.8) | 33.2-295 |
| Phosphorus (mg) | 688 | (235.1) | 163.8-1349.4 |
| Potassium (mg) | 1281.2 | (548.7) | 131-3268.1 |
| Selenium (mcg) | 55.1 | (25.9) | 6.2-150 |
| Valine (g) | 0.5 | (0.4) | 0-1.5 |
| Vitamin A (mcg) | 453.3 | (344.5) | 30.6-2597.6 |
| Vitamin B1 (mcg) | 0.8 | (0.4) | 0-1.7 |
| Vitamin B2 (mcg) | 1.3 | (0.5) | 0.3-2.5 |
| Vitamin B6 (mcg) | 1 | (0.6) | 0.2-4 |
| 3′SL (mg) | 15.3 | (17.3) | 0-54.6 |
| Zinc (mg) | 6.2 | (2.8) | 0.2-13 |

Age window 21-30 months

| | | | |
|---|---|---|---|
| alpha_lactalbumin (g) | 0.3 | (0.2) | 0-0.9 |
| Cryptoxanthin (mg) | 117.3 | (102.6) | 0.4-387 |
| Gangliosides (mg) | 3.1 | (2.6) | 0.5-12 |
| Oligofructose | 1.6 | (1.5) | 0-5.2 |
| PFA 18:4 (Octadecatetraenoic acid) | 4 | (5.8) | 0-26 |
| (mg) | | | |
| PFA 20:4 (Eicosatetraenoic acid) | 0.1 | (0.1) | 0-0.3 |
| (g) | | | |
| PFA 22:5 (Docosapentaenoic acid) | 4.1 | (6.3) | 0-42 |
| (mg) | | | |
| Phosphatidylcholine (g) | 0.5 | (0.7) | 0-3.5 |
| Phosphatidylinositol (mg) | 9.8 | (11.8) | 0-47.5 |
| Sphingomyelin (mg) | 7 | (8.4) | 0-43.9 |
| 3′SL (mg) | 20.6 | (20.8) | 0-90.5 |
| 6′SL (mg) | 4 | (4) | 0.1-18.3 |

Age window 31-60 months

| | | | |
|---|---|---|---|
| Alpha-carotene (mcg) | 276.7 | (264.1) | 0-1669.5 |
| alpha_lactalbumin (g) | 0.2 | (0.2) | 0-0.8 |
| Alpha tocopherol (mg) | 6 | (2.8) | 0-17.9 |
| Gangliosides (mg) | 3.1 | (2.5) | 0.1-13 |
| Histidine (mg) | 0.3 | (0.2) | 0-0.9 |
| Isoleucine (mg) | 0.5 | (0.2) | 0-1.7 |
| Leucine (mg) | 1.1 | (0.7) | 0-2.9 |
| Lysine (mg) | 0.8 | (0.4) | 0-2.3 |
| MFA 20:1 (Eicosenoic acid) (g) | 0.1 | (0.1) | 0-0.5 |
| PFA 18:4 (Octadecatetraenoic acid) | 6.8 | (10.1) | 0-56 |
| (mg) | | | |
| PFA 20:5 (Eicosapentaenoic acid) | 8.3 | (15.4) | 0-127 |
| (mg) | | | |
| PFA 22:5 (Docosapentaenoic acid) | 10.1 | (8.6) | 0-37 |
| (mg) | | | |
| PFA 22:6 (Docosahexaenoic acid) | 32.1 | (46.2) | 0-255 |
| (mg) | | | |
| Phosphatidylcholine (g) | 0.6 | (0.6) | 0-3.2 |
| Phosphatidylethanolamine (g) | 0.1 | (0.1) | 0-0.7 |
| Phosphorus (mg) | 894.2 | (253.4) | 32.5-1473.6 |
| Phosphatidylinositol (mg) | 16.5 | (22.1) | 0-168.3 |
| Phosphatidylserine (mg) | 18.3 | (34.7) | 0-293.4 |
| Sphingomyelin (mg) | 9.7 | (11.4) | 0.1-76.6 |
| Tryptophan (g) | 0.1 | (0.1) | 0-0.6 |
| Valine (g) | 0.7 | (0.3) | 0-2 |
| 3′SL (mg) | 11.7 | (15) | 0-81.6 |
| 6′SL (mg) | 2.7 | (2.9) | 0-16.5 |

^{a}Abbreviations: SD; standard deviation, g; grams, mg; milligrams, mcg; micrograms.

In addition, five nutrients were identified across the windows with a positive and significant association to myelin according to the linear model (Table 5). The variables selected by the random forest model and sorted by average importance for nutrients across ages were as follows: log Age; Gangliosides (mg); Sphingomyelin (mg); 18:2, Octadecadienoic acid (g); 22:6 n-3, Docosahexaenoic acid [DHA](g); Phosphatidylcholine (g); Phosphatidylinositol (g); 20:4, Eicosatetraenoic acid (g); Fatty acids, total saturated (g); 3′-SL (mg); Phosphorus (mg); Fatty acids, total polyunsaturated (g); Food Folate (μg); Isoleucine (g); Methionine (g); Total Fat (g); Total Phospholipids (g); Carotene, beta (mcg); Serine (g); Calcium (mg); Phosphatidylserine (g).

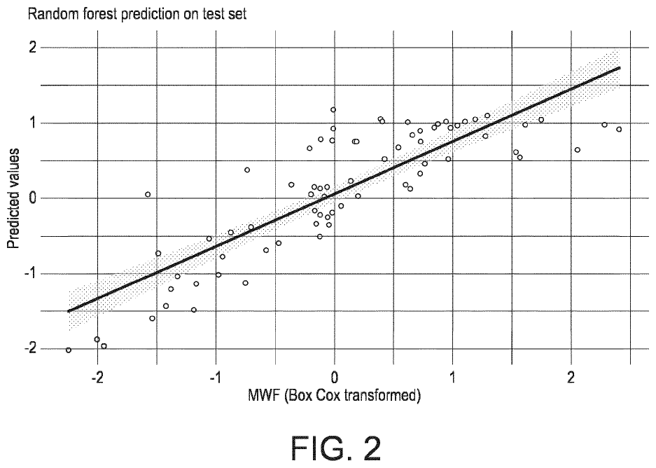

FIG. 2 compares the values of the transformed MWF with the values predicted by the random forest, on a test set. The Pearson correlation between predictions and original values is 0.88 (95% confidence interval 0.81-0.92), indicating that the model predicts myelin values appropriately.

TABLE 5
Nutrients across ages with positive
and significant myelin associations

| term | estimate | std.error | p.value | nutrient |
|---|---|---|---|---|
| (Inercept) | −0.168 | 0.012 | <0.001 | Lysine |
| nutIntake | 0.033 | 0.017 | 0.048 | |
| logAge | 0.036 | 0.002 | <0.001 | |
| nutIntake*logAge | −0.005 | 0.002 | 0.049 | |
| (Intercept) | −0.168 | 0.008 | 0.000 | 3′SL |
| nutIntake | 0.002 | 0.000 | <0.001 | |
| logAge | 0.036 | 0.001 | <0.001 | |
| nutIntake*logAge | 0.000 | 0.000 | <0.001 | |
| (Intercept) | −0.168 | 0.008 | 0.000 | 6′SL |
| nutIntake | 0.009 | 0.002 | <0.001 | |
| logAge | 0.036 | 0.001 | <0.001 | |
| nutIntake*logAge | −0.001 | 0.000 | <0.001 | |
| (Intercept) | −0.156 | 0.008 | <0.001 | Oligofructose |
| nutIntake | 0.004 | 0.002 | 0.039 | |
| logAge | 0.034 | 0.001 | <0.001 | |
| nutIntake*logAge | −0.001 | 0.000 | 0.043 | |
| (Intercept) | −0.166 | 0.010 | 0.000 | α-lactalbumin |
| nutIntake | 0.097 | 0.040 | 0.015 | |
| logAge | 0.036 | 0.001 | <0.001 | |
| nutIntake*logAge | −0.014 | 0.006 | 0.016 | |

### DISCUSSION

The inventors describe for the first time nutrient-myelin windows and their dynamics in well-nourished young children applying myelin imaging to identifying windows of sensitivity for brain stage-related nutrients. While four of the evaluated nutrients were significantly associated with myelin development at all age windows up to 5 years, most nutrients followed a dynamic association pattern overtime. The inventors have identified three nutrient-myelin windows across the age range of 6 to 60 months that follow previously defined brain development dynamics of a steep increase in myelin in the first 1.5 postnatal years, followed by a transition to slower growth around 2 years of age, and then continued slower myelin increase at 3 to 5 years.

Relations between brain growth and emerging skills or behaviors as well as between brain growth and influencing factors have been well described. The dynamic relations between nutrition, brain and cognitive development have been highlighted by Georgieff. The inventors' findings add to these ideas by supporting new hypotheses and observational data for age and brain stage appropriate nutritional opportunities, in alignment with the dynamic needs of the developing brain. Timing is critical for both, nutritional deficiencies as well as effective support and nutritional intervention during development. Understanding sensitive periods for the specific link between nutrients and brain development are foundational for developing effective nutritional solutions and credible recommendations for brain nutrients. Interestingly, all classes of nutrients showed dynamic patterns and sensitive windows. Micronutrients and fatty acids appear particularly relevant in the first 1.5 years of life during the time of steep myelin increase, while both nutrient classes phase out during the brain development window of myelin rate change, fatty acids phase back in after 2.5 years of age when myelin increase continues at a slower rate. This may indicate the relevance of fatty acids in myelin accumulation. The formation of the myelin sheath requires high levels of fatty acid and lipid synthesis as well as the uptake of extracellular fatty acids. In contrast, the results show that amino acids gain importance in toddlerhood when the myelin spurt slows down and transitions to slower continued growth. Amino acids may be more indirectly relevant for developmental myelination. Amino acid abnormalities have been linked to disturbed protein synthesis which may affect myelin synthesis. Dietary essential amino acids like tryptophan and tyrosine may play a role in neurodevelopment, including motor and sensory functions.

For children aged 6 to 20 months (window 1), a predominant contribution to higher myelin levels came from fatty acids and micronutrients (vitamins and minerals). In contrast, for children aged 20 to 30 months (window 2) mostly amino acids contributed to myelination and for children aged 30 to 60 months (window 3) mixed contributions from both fatty acids and amino acids were identified. In addition, age-adjusted models exploring nutrient-myelin associations across age windows suggest the oligosaccharides 3′SL and 6′SL, lysine, alpha-lactalbumin and oligofructose to be linked with higher myelin levels. Little is known about the role of these nutrients in developmental myelination. But 6′SL has been recently shown to impact myelination in a preclinical study, potentially via a reduction in sialylated binding targets for the myelin associated glycoprotein.

1: An age-tailored kit of nutrient blend compositions to be sequentially administered to a young individual to promote myelination of targeted brain regions, the kit comprising at least two of the following compositions:
a) a nutrient blend composition A to be administered to a young individual at age from 0 to 20 months, the nutrient blend composition A comprising at least three ingredients selected from the group consisting of vitamin B1, vitamin B2, vitamin B6, zinc, iron, copper, histidine, isoleucine, lysine, leucine, vitamin A, folate, magnesium, phosphorus, potassium, selenium, 3′-SL, alanine, cystine, glutamic acid, phenylalanine, proline, serine, threonine, tyrosine, and valine;
b) a nutrient blend composition B to be administered to a young individual at age from 20 to 30 months, the nutrient blend composition B comprising at least three ingredients selected from the group consisting of DPA, ARA, sphingomyelin, phosphatidylcholine, octadecatetraenoic acid, oligofructose, 6′-SL, stearidonic acid, beta cryptoxanthin, alpha lactalbumin, gangliosides, phosphatidylinositol, 3′-SL, and eicosatetraenoic acid; and
c) a nutrient blend composition C to be administered to a young individual at age from 30 to 66 months, the nutrient blend composition C comprising at least three ingredients selected from the group consisting of DHA, sphingomyelin, phosphatidylcholine, histidine, isoleucine, leucine, valine, alpha carotene, folic acid, alpha lactalbumin, gangliosides, phosphatidylinositol, phosphatidylethanolamine, eicosenoic acid, stearidonic acid, DPA, 3′-SL, 6′-SL, alanine, arginine, aspartic acid, cystine, glutamic acid, glycine, lysine, methionine, phenylalanine, proline, serine, threonine, tryptophan, tyrosine, alpha tocopherol, octadecatetraenoic acid, eicosapentaenoic acid, phosphorus, and phosphatidylserine.

2: The kit of claim 1, wherein;
a) the nutrient blend composition A is to be administered to a young individual at age from 6 to 20 months;
b) the nutrient blend composition B is to be administered to a young individual at age from 20 to 30 months;
c) the nutrient blend composition C is to be administered to a young individual at age from 30 to 60 months.

3: The kit of claim 1, wherein:
a) the nutrient blend composition A comprises at least three ingredients selected from the group consisting of vitamin B1, vitamin B2, vitamin B6, zinc, iron, copper, histidine, isoleucine, lysine, and leucine;
b) the nutrient blend composition B comprises at least three ingredients selected from the group consisting of DPA, ARA, sphingomyelin, phosphatidylcholine, octadecatetraenoic acid, oligofructose, and 6′-SL;
c) the nutrient blend composition C comprises at least three ingredients selected from the group consisting of DHA, sphingomyelin, phosphatidylcholine, histidine, isoleucine, leucine, and valine.

4: The kit of claim 1, wherein:
a) the nutrient blend composition A comprises at least three ingredients selected from the group consisting of:
vitamin B1 in a daily amount of at least about 0.8 mg;
vitamin B2 in a daily amount of at least about 1 mg;
vitamin B6 in a daily amount of at least about 0.9 mg;
zinc in a daily amount of at least about 6 mg;
iron in a daily amount of at least about 8 mg;
copper in a daily amount of at least about 0.6 mg;
histidine in a daily amount of at least about 0.2 g;
isoleucine in a daily amount of at least about 0.3 g;
lysine in a daily amount of at least about 0.4 g;
leucine in a daily amount of at least 0.6 g;
vitamin A in a daily amount of at least about 400 mg;
folate in a daily amount of at least about 168 mg;
magnesium in a daily amount of at least about 143 mg;
phosphorus in a daily amount of at least about 688 mg;
potassium in a daily amount of at least about 1267 mg;
selenium in a daily amount of at least about 51 μg;
3′-SL in a daily amount of at least about 4 mg;
alanine in a daily amount of at least about 0.3 g;
cystine in a daily amount of at least about 0.07 g;
glutamic acid in a daily amount of at least about 2 g;
phenylalanine in a daily amount of at least about 0.4 g;
proline in a daily amount of at least about 0.6 g;
serine in a daily amount of at least about 0.4 g;
threonine in a daily amount of at least about 0.3 g;
tyrosine in a daily amount of at least about 0.3 g; and
valine in a daily amount of at least about 0.5 g;
b) the nutrient blend composition B comprises at least three ingredients selected from the group consisting of:
DPA in a daily amount of at least about 3 mg;
ARA in a daily amount of at least about 0.06 g;
sphingomyelin in a daily amount of at least about 3 mg;
phosphatidylcholine in a daily amount of at least about 0.3 g;
octadecatetraenoic acid in a daily amount of at least about 3 mg;
oligofructose in a daily amount of at least about 1 g;
6′-SL in a daily amount of at least about 3 mg;
stearidonic acid in a daily amount of at least about 3 mg;
beta cryptoxanthin in a daily amount of at least about 93 mg;
alpha lactalbumin in a daily amount of at least about 0.3 mg;
gangliosides in a daily amount of at least about 2 mg;
phosphatidylinositol in a daily amount of at least about 4 mg;
3′-SL in a daily amount of at least about 16 mg; and
eicosatetraenoic acid in a daily amount of at least about 0.1 g; and
c) the nutrient blend composition C comprises at least three ingredients selected from the group consisting of:
DHA in a daily amount of at least about 0.01 g;
sphingomyelin in a daily amount of at least about 5 mg;
phosphatidylcholine in a daily amount of at least about 0.4 g;
histidine in a daily amount of at least about 0.3 g;
isoleucine in a daily amount of at least about 0.5 g;
leucine in a daily amount of at least about 1 g;
valine in a daily amount of at least about 0.7 g;
alpha carotene in a daily amount of at least about 214 mg;
folate in daily amount of at least about 251 mg;
alpha lactalbumin in a daily amount of at least about 0.1 mg;
gangliosides in a daily amount of at least about 2 mg;
phosphatidylinositol in a daily amount of at least about 0.01 g;
phosphatidylethnolamine in a daily amount of at least about 0.05 g;
eicosenoic acid in a daily amount of at least about 0.1 g;
stearidonic acid in daily amount of at least about 3 mg;
DPA in a daily amount of at least about 8 mg;
3′-SL in a daily amount of at least about 6 mg;
6′SL in a daily amount of at least about 2 mg;
alanine in a daily amount of at least about 0.5 g;
arginine in a daily amount of at least about 0.5 g;
aspartic acid in a daily of at least about 1 g;
cystine in a daily amount of at least about 0.1 g;
glutamic acid in a daily of at least about 2 g;
glycine in a daily amount of at least about 0.3 g;
lysine in a daily amount of at least about 0.7 g;
methionine in a daily amount of at least about 0.2 g;
phenylalanine in a daily amount of at least about 0.6 g;
proline in a daily amount of at least about 1 g;
serine in a daily amount of at least about 0.6 g;
threonine in a daily amount of at least about 0.5 g;
tryptophan in a daily amount of at least about 0.1 g;
tyrosine in a daily amount of at least about 0.5 g;
alpha tocopherol in a daily amount of at least about 6 mg;
octadecatetraenoic acid in a daily amount of at least about 6.8 mg;
eicosapentaenoic acid in a daily amount of at least about 8 mg;
phosphorus in a daily amount of at least about 894 mg; and
phosphatidylserine in a daily amount of at least about 18 mg.

5: The kit of claim 1, wherein
a) the nutrient blend composition A comprises at least three ingredients selected from the group consisting of:
vitamin B1 in a daily amount ranging from about 0.8 to about 2 mg;
vitamin B2 in a daily amount ranging from about 1 to about 3 mg;
vitamin B6 in a daily amount ranging from about 0.9 to about 5 mg;
zinc in a daily amount ranging from about 6 to about 13 mg;
iron in a daily amount ranging from about 8 to about 29 mg;
copper in a daily amount ranging from about 0.6 to about 1.1 mg;
histidine in a daily amount ranging from about 0.2 to about 1 g;
isoleucine in a daily amount ranging from about 0.3 to about 2 g;
lysine in a daily amount ranging from about 0.4 to about 3 g;
leucine in a daily amount ranging from about 0.6 to about 4 g;
vitamin A in a daily amount ranging from about 400 to about 2600 mg;
folate in a daily amount ranging from about 168 to about 460 mg;
magnesium in a daily amount ranging from about 143 to about 369 mg;
phosphorus in a daily amount ranging from about 688 to about 1687 mg;
potassium in a daily amount ranging from about 1267 to about 4085 mg;
selenium in a daily amount ranging from about 51 to about 150 μg;
3′-SL in a daily amount ranging from about 4 to about 68 mg;
alanine in a daily amount ranging from about 0.3 to about 2 g;
cystine in a daily amount ranging from about 0.07 to about 0.4 g;
glutamic acid in a daily amount ranging from about 2 to about 10 g;
phenylalanine in a daily amount ranging from about 0.4 to about 2 g;
proline in a daily amount ranging from about 0.6 to about 3 g;
serine in a daily amount ranging from about 0.4 to about 2 g;
threonine in a daily amount ranging from about 0.3 g to about 2 g;
tyrosine in a daily amount ranging from about 0.3 to about 2 g; and
valine in a daily amount ranging from about 0.5 to about 2 g;
b) the nutrient blend composition B comprises at least three ingredients selected from the group consisting of:
DPA in a daily amount ranging from about 3 mg to about 53 mg;
ARA in a daily amount ranging from about 0.06 to about 0.4 g;
sphingomyelin in a daily amount ranging from about 3 to about 55 mg;
phosphatidylcholine in a daily amount ranging from about 0.3 to about 5 g;
octadecatetraenoic acid in a daily amount ranging from about 3 to about 33 mg;
oligofructose in a daily amount ranging from about 1 to about 7 g;
6′-SL in a daily amount ranging from about 3 to about 23 mg;
stearidonic acid in a daily amount ranging from about 3 to about 41 mg;
beta cryptoxanthin in a daily amount ranging from about 93 to about 484 mg;
alpha lactalbumin in a daily amount ranging from about 0.3 to about 1 mg;
gangliosides in a daily amount ranging from about 2 to about 15 mg;
phosphatidylinositol in a daily amount ranging from about 4 to about 59 mg; and
3′-SL in a daily amount ranging from about 16 to about 113 mg; and
eicosatetraenoic acid in a daily amount ranging from about 0.1 to about 0.3 g; and
c) the nutrient blend composition C comprises at least three ingredients chosen from the group consisting of:
DHA in a daily amount ranging from about 0.01 to about 0.3 g;
sphingomyelin in a daily amount ranging from about 5 to about 96 mg;
phosphatidylcholine in a daily amount ranging from about 0.4 to about 4 g;
histidine in a daily amount ranging from about 0.3 to about 1 g;
isoleucine in a daily amount ranging from about 0.5 to about 2 g;
leucine in a daily amount ranging from about 1 to about 4 g;
valine in a daily amount ranging from about 0.7 to about 3 g;
alpha carotene in a daily amount ranging from about 214 to about 2087 mg;
folate in daily amount ranging from about 251 to about 400 mg;
alpha lactalbumin in a daily amount ranging from about 0.1 to about 1 mg;
gangliosides in a daily amount ranging from about 2 to about 16 mg;
phosphatidylinositol in a daily amount ranging from about 0.01 to about 0.2 g;
phosphatidylethnolamine in a daily amount ranging from about 0.05 to about 0.9 g;
eicosenoic acid in a daily amount ranging from about 0.1 to about 0.5 g;
stearidonic acid in daily amount ranging from about 3 to about 70 mg;
DPA in a daily amount ranging from about 8 to about 46 mg;
3′-SL in a daily amount ranging from 6 to about 102 mg;
6′SL in a daily amount ranging from about 2 to about 21 mg;
alanine in a daily amount ranging from about 0.5 to about 2 g;
arginine in a daily amount ranging from about 0.5 to about 2 g;
aspartic acid in a daily amount ranging from about 1 to about 4 g;
cystine in a daily amount ranging from about 0.1 to about 0.6 g;
glutamic acid in a daily amount ranging from about 2 to about 11 g;
glycine in a daily amount ranging from about 0.3 to about 2 g;
lysine in a daily amount ranging from about 0.7 to about 3 g;
methionine in a daily amount ranging from about 0.2 to about 1 g;
phenylalanine in a daily amount ranging from about 0.6 to about 2 g;
proline in a daily amount ranging from about 1 to about 4 g;
serine in a daily amount ranging from about 0.6 to about 2 g;
threonine in a daily amount ranging from about 0.5 to about 2 g;
tryptophan in a daily amount ranging from about 0.1 to about 0.8 g;
tyrosine in a daily amount ranging from about 0.5 to about 2 g;
alpha tocopherol in a daily amount ranging from about 6 to about 18 mg;
octadecatetraenoic acid in a daily amount ranging from about 6.8 to about 56 mg;
eicosapentaenoic acid in a daily amount ranging from about 8 to about 127 mg;
phosphorus in a daily amount ranging from about 894 to about 1475 mg;
phosphatidylserine in a daily amount ranging from about 18 to about 294 mg.

6: The kit of claim 1, wherein:
a) the nutrient blend composition A comprises at least three ingredients selected from the group consisting of:
vitamin B1 in an amount of at least about 0.2 mg per 100 ml of liquid;
vitamin B2 in an amount of at least about 0.2 mg per 100 ml of liquid;
vitamin B6 in an amount of at least about 0.2 mg per 100 ml of liquid;
zinc in an amount of at least about 1 mg per 100 ml of liquid;
iron in an amount of at least about 2 mg per 100 ml of liquid;
copper in an amount of at least about 0.1 mg per 100 ml of liquid;
histidine in an amount of at least about 0.04 g per 100 ml of liquid;
isoleucine in an amount of at least about 0.06 g per 100 ml of liquid;
lysine in an amount of at least about 0.08 g per 100 ml of liquid;
leucine in an amount of at least about 0.1 g per 100 ml of liquid;
vitamin A in an amount of at least about 81 μg per 100 ml of liquid;
folate in an amount of at least about 34 μg per 100 ml of liquid;
magnesium in an amount of at least about 29 mg per 100 ml of liquid;
phosphorus in an amount of at least about 141 mg per 100 ml of liquid;
potassium in an amount of at least about 258 mg per 100 ml of liquid;
selenium in an amount of at least about 10 μg per 100 ml pf liquid;
3′-SL in an amount of at least about 0.8 mg per 100 ml of liquid;
alanine in an amount of at least about 0.06 g per 100 ml of liquid;
cystine in an amount of at least about 0.01 g per 100 ml of liquid;
glutamic acid in an amount of at least about 0.4 g per 100 ml of liquid;
phenylalanine in an amount of at least about 0.08 g per 100 ml of liquid;
proline in an amount of at least about 0.1 g per 100 ml of liquid;
serine in an amount of at least about 0.08 g per 100 ml of liquid;
threonine in an amount of at least about 0.06 g per 100 ml of liquid;
tyrosine in an amount of at least about 0.06 g per 100 ml of liquid; and
valine in an amount of at least about 0.1 g per 100 ml of liquid;
b) the nutrient blend composition B comprises at least three ingredients selected from the group consisting of:
DPA in an amount of at least about 0.6 mg per 100 ml of liquid;
ARA in an amount of at least about 0.01 g per 100 ml of liquid;
sphingomyelin in an amount of at least about 0.6 mg per 100 ml of liquid;
phosphatidylcholine in an amount of at least about 0.06 g per 100 ml of liquid;
octadecatetraenoic acid in an amount of at least about 0.6 mg per 100 ml of liquid;
oligofructose in an amount of at least about 0.2 g per 100 ml of liquid;
6′-SL in an amount of at least about 0.6 mg per 100 ml of liquid;
stearidonic acid in an amount of at least about 0.6 mg per 100 ml of liquid;
beta cryptoxanthin in an amount of at least about 19 mg per 100 ml of liquid;
alpha lactalbumin in an amount of at least about 0.06 mg per 100 ml of liquid;
gangliosides in an amount of at least about 0.4 mg per 100 ml of liquid;
phosphatidylinositol in an amount of at least about 0.8 mg per 100 ml of liquid; and
3′-SL in an amount of at least about 3 mg per 100 ml of liquid; and
eicosatetraenoic acid in an amount of at least about 0.01 g per 100 ml of liquid; and
c) the nutrient blend composition C comprises at least three ingredients selected from the group consisting of:
DHA in an amount of at least about 0.01 g per 100 ml of liquid;
sphingomyelin in an amount of at least about 1 mg per 100 ml of liquid;
phosphatidylcholine in an amount of at least about 0.08 g per 100 ml of liquid;
histidine in an amount of at least about 0.06 g per 100 ml of liquid;
isoleucine in an amount of at least about 0.1 g per 100 ml of liquid;
leucine in an amount of at least about 0.2 g per 100 ml of liquid;
valine in an amount of at least about 0.1 g per 100 ml of liquid;
alpha carotene in an amount of at least about 43 mg per 100 ml of liquid;
folate in an amount of at least about 51 μg per 100 ml of liquid;
alpha lactalbumin in an amount of at least about 0.02 mg per 100 ml of liquid;
gangliosides in an amount of at least about 0.4 mg per 100 ml of liquid;
phosphatidylinositol in an amount of at least about 0.01 g per 100 ml of liquid;
phosphatidylethanolamine in an amount of at least about 0.01 g per 100 ml of liquid;
eicosenoic acid in an amount of at least about 0.02 g per 100 ml of liquid;
stearidonic acid in an amount of at least about 0.6 mg per 100 ml of liquid;
DPA in an amount of at least about 2 mg per 100 ml of liquid;
3′-SL in an amount of at least about 1 mg per 100 ml of liquid;
6′-SL in an amount of at least about 0.4 mg per 100 ml of liquid;
alanine in an amount of at least about 0.1 g per 100 ml of liquid;
arginine in an amount of at least about 0.1 g per 100 ml of liquid;
aspartic acid in an amount of at least about 0.2 g per 100 ml of liquid;
cystine in an amount of at least about 0.02 g per 100 ml of liquid;
glutamic acid in an amount of at least about 0.4 g per 100 ml of liquid;
glycine in an amount of at least about 0.06 g per 100 ml of liquid;
lysine in an amount of at least about 0.1 g per 100 ml of liquid;
methionine in an amount of at least about 0.04 g per 100 ml of liquid;
phenylalanine in an amount of at least about 0.1 g per 100 ml of liquid;
proline in an amount of at least about 0.2 g per 100 ml of liquid;
serine in an amount of at least about 0.1 g per 100 ml of liquid;
threonine in an amount of at least about 0.1 g per 100 ml of liquid;
tryptophan in an amount of at least about 0.02 g per 100 ml of liquid;
tyrosine in an amount of at least about 0.1 g per 100 ml of liquid;
alpha tocopherol in an amount of at least about 1.2 mg per 100 ml of liquid;
octadecatetraenoic acid in an amount of at least about 1.3 mg per 100 ml of liquid;
eicosapentaenoic acid in an amount of at least about 1.7 mg per 100 ml of liquid;
phosphorus in an amount of at least about 0.1 g per 100 ml of liquid; and
phosphatidylserine in an amount of at least about 0.01 g per 100 ml of liquid.

7: The kit of claim 1, wherein:
a) the nutrient blend composition A comprises at least three ingredients selected from the group consisting of:
vitamin B1 in an amount ranging from about 0.2 to about 0.4 mg per 100 ml of liquid;
vitamin B2 in an amount ranging from about 0.2 to about 0.6 mg per 100 ml of liquid;
vitamin B6 in an amount ranging from about 0.2 to about 1 mg per 100 ml of liquid;
zinc in an amount ranging from about 1 to about 2.66 mg per 100 ml of liquid;
iron in an amount ranging from about 2 to about 6 mg per 100 ml of liquid;
copper in an amount ranging from about 0.1 to about 0.2 mg per 100 ml of liquid;
histidine in an amount ranging from about 0.04 to about 0.2 g per 100 ml of liquid;
isoleucine in an amount ranging from about 0.06 to about 0.4 g per 100 ml of liquid;
lysine in an amount ranging from about 0.08 to about 0.6 g per 100 ml of liquid;
leucine in an amount ranging from about 0.1 to about 0.8 g per 100 ml of liquid;
vitamin A in an amount ranging from about 81 to about 532 μg per 100 ml of liquid;
folate in an amount ranging from about 34 to about 94 μg per 100 ml of liquid;
magnesium in an amount ranging from about 29 to about 75 mg per 100 ml of liquid;
phosphorus in an amount ranging from about 141 to about 343 mg per 100 ml of liquid;
potassium in an amount ranging from about 258 to about 830 mg per 100 ml of liquid;
selenium in an amount ranging from about 10 to about 31 μg per 100 ml of liquid;
3′-SL in an amount ranging from about 0.8 to about 14 mg per 100 ml of liquid;
alanine in an amount ranging from about 0.06 to about 0.4 g per 100 ml of liquid;
cystine in an amount ranging from about 0.01 to about 0.08 g per 100 ml of liquid;
glutamic acid in an amount ranging from about 0.4 to about 2 g per 100 ml of liquid;
phenylalanine in an amount ranging from about 0.08 to about 0.4 g per 100 ml of liquid;
proline in an amount ranging from about 0.1 to about 0.6 g per 100 ml of liquid;
serine in an amount ranging from about 0.08 to about 0.4 g per 100 ml of liquid;
threonine in an amount ranging from about 0.06 to about 0.4 g per 100 ml of liquid;
tyrosine in an amount ranging from about 0.06 to about 0.4 g per 100 ml of liquid; and
valine in an amount ranging from about 0.1 to about 0.4 g of per 100 ml of liquid;
b) the nutrient blend composition B comprises at least three ingredients selected from the group consisting of:
DPA in an amount ranging from about 0.6 to about 11 mg per 100 ml of liquid;
ARA in an amount ranging from about 0.01 to about 0.08 g per 100 ml of liquid;
sphingomyelin in an amount ranging from about 0.6 to about 11 mg per 100 ml of liquid;
phosphatidylcholine in an amount ranging from about 0.06 to about 1 g per 100 ml of liquid;
octadecatetraenoic acid in an amount ranging from about 0.6 to about 7 mg per 100 ml of liquid;
oligofructose in an amount ranging from about 0.2 to about 2 g per 100 ml of liquid;
6′-SL in an amount ranging from about 0.6 to about 5 mg per 100 ml of liquid;
stearidonic acid in an amount ranging from about 0.6 to about 9 mg per 100 ml of liquid;
beta cryptoxanthin in an amount ranging from about 19 to about 98 mg per 100 ml of liquid;
alpha lactalbumin in an amount ranging from about 0.06 to about 0.2 mg per 100 ml of liquid;
gangliosides in an amount ranging from about 0.4 to about 3 mg per 100 ml of liquid;
phosphatidylinositol in an amount ranging from about 0.8 to about 12 mg per 100 ml of liquid; and
3′-SL in an amount ranging from about 3 to about 23 mg per 100 ml of liquid; and
eicosatetraenoic acid in an amount ranging from about 0.02 to about 0.06 g per 100 ml of liquid; and
c) the nutrient blend composition C comprises at least three ingredients selected from the group consisting of:
DHA in an amount ranging from about 0.01 to about 0.06 g per 100 ml of liquid;
sphingomyelin in an amount ranging from about 1 to about 20 mg per 100 ml of liquid;
phosphatidylcholine in an amount ranging from about 0.08 to about 0.8 g per 100 ml of liquid;
histidine in an amount ranging from about 0.06 to about 0.2 g per 100 ml of liquid;
isoleucine in an amount ranging from about 0.1 to about 0.4 g per 100 ml of liquid;
leucine in an amount ranging from about 0.2 to about 0.8 g per 100 ml of liquid;
valine in an amount ranging from about 0.1 to about 0.6 g per 100 ml of liquid;
alpha carotene in an amount ranging from about 43 to about 424 mg per 100 ml of liquid;
folate in an amount ranging from about 51 to about 82 μg per 100 ml of liquid;
alpha lactalbumin in an amount ranging from about 0.02 to about 0.2 mg per 100 ml of liquid;
gangliosides in an amount ranging from about 0.4 to about 3 mg per 100 ml of liquid;
phosphatidylinositol in an amount ranging from about 0.01 to about 0.04 g per 100 ml of liquid;
phosphatidylethanolamine in an amount ranging from about 0.01 to about 0.2 g per 100 ml of liquid;
eicosenoic acid in an amount ranging from about 0.02 to about 0.11 g per 100 ml of liquid;
stearidonic acid in an amount ranging from about 0.6 to about 14 mg per 100 ml of liquid;
DPA in an amount ranging from about 2 to about 9 mg per 100 ml of liquid;
3′-SL in an amount ranging from about 1 to about 21 mg per 100 ml of liquid;
6′-SL in an amount ranging from about 0.4 to about 4 mg per 100 ml of liquid;
alanine in an amount ranging from about 0.1 to about 0.4 g per 100 ml of liquid;
arginine in an amount ranging from about 0.1 to about 0.4 g per 100 ml of liquid;
aspartic acid in an amount ranging from about 0.2 to about 0.8 g per 100 ml of liquid;
cystine in an amount ranging from about 0.02 to about 0.1 g per 100 ml of liquid;
glutamic acid in an amount ranging from about 0.4 to about 2 g per 100 ml of liquid;
glycine in an amount ranging from about 0.06 to about 0.4 g per 100 ml of liquid;
lysine in an amount ranging from about 0.1 to about 0.6 g per 100 ml of liquid;
methionine in an amount ranging from about 0.04 to about 0.2 g per 100 ml of liquid;
phenylalanine in an amount ranging from about 0.1 to about 0.4 g per 100 ml of liquid;
proline in an amount ranging from about 0.2 to about 0.8 g per 100 ml of liquid;
serine in an amount ranging from about 0.1 to about 0.6 g per 100 ml of liquid;
threonine in an amount ranging from about 0.1 to about 0.4 g per 100 ml of liquid;
tryptophan in an amount ranging from about 0.02 to about 0.2 g per 100 ml of liquid; and
tyrosine in an amount ranging from about 0.1 to about 0.4 g per 100 ml of liquid;
alpha tocopherol in an amount ranging from about 1.2 to about 3.7 mg per 100 ml of liquid;
octadecatetraenoic acid in an amount ranging from about 1.3 to about 116 mg per 100 ml of liquid;
eicosapentaenoic acid in an amount ranging from about 1.7 to about 26 mg per 100 ml of liquid;
phosphorus in an amount ranging from about 0.1 to about 300 mg per 100 ml of liquid; and
phosphatidylserine in an amount ranging from about 0.01 to about 60 mg per 100 ml of liquid.

8: The kit of claim 1, wherein each of the nutrient blend compositions A, B and C comprise at least four ingredients selected from the group.

9: The kit of claim 1, wherein each of said compositions differ one from the other, in at least one of the ingredients.

10: The kit of claim 1, wherein the young individual is a human, a pet, an infant, a young child or a child.

11: A method for promoting brain myelination of targeted brain regions of a young individual, the method comprising sequentially administering a kit to the young individual, the kit comprising at least two of the following compositions:
a) a nutrient blend composition A administered to the young individual at age from 0 to 20 months, the nutrient blend composition A comprising at least three ingredients selected from the group consisting of vitamin B1, vitamin B2, vitamin B6, zinc, iron, copper, histidine, isoleucine, lysine, leucine, vitamin A, folate, magnesium, phosphorus, potassium, selenium, 3′-SL, alanine, cystine, glutamic acid, phenylalanine, proline, serine, threonine, tyrosine, and valine;
b) a nutrient blend composition B administered to the young individual at age from 20 to 30 months, the nutrient blend composition B comprising at least three ingredients selected from the group consisting of DPA, ARA, sphingomyelin, phosphatidylcholine, octadecatetraenoic acid, oligofructose, 6′-SL, stearidonic acid, beta cryptoxanthin, alpha lactalbumin, gangliosides, phosphatidylinositol, 3′-SL, and eicosatetraenoic acid; and
e) a nutrient blend composition C administered to the young individual at age from 30 to 66 months, the nutrient blend composition C comprising at least three ingredients selected from the group consisting of DHA, sphingomyelin, phosphatidylcholine, histidine, isoleucine, leucine, valine, alpha carotene, folic acid, alpha lactalbumin, gangliosides, phosphatidylinositol, phosphatidylethanolamine, eicosenoic acid, stearidonic acid, DPA, 3′-SL, 6′-SL, alanine, arginine, aspartic acid, cystine, glutamic acid, glycine, lysine, methionine, phenylalanine, proline, serine, threonine, tryptophan, tyrosine, alpha tocopherol, octadecatetraenoic acid, eicosapentaenoic acid, phosphorus, and phosphatidylserine.

12: The method of claim 11, wherein the targeted brain regions comprise at least one region of the social brain.

13: A staged feeding regimen of a young individual, the staged feeding regimen comprising:
a) administering a nutrient blend composition A to the young individual at age from 0 to 20 months,
the nutrient blend composition A comprising at least three ingredients selected from the group consisting of vitamin B1, vitamin B2, vitamin B6, zinc, iron, copper, histidine, isoleucine, lysine, leucine, vitamin A, folate, magnesium, phosphorus, potassium, selenium, 3′-SL, alanine, cystine, glutamic acid, phenylalanine, proline, serine, threonine, tyrosine, and valine;
b) administering a nutrient blend composition B to the young individual at age from 20 to 30 months,
the nutrient blend composition B comprising at least three ingredients selected from the group consisting of DPA, ARA, sphingomyelin, phosphatidylcholine, octadecatetraenoic acid, oligofructose, 6′-SL, stearidonic acid, beta cryptoxanthin, alpha lactalbumin, gangliosides, phosphatidylinositol, 3′-SL, and eicosatetraenoic acid; and
c) administering a nutrient blend composition C to the young individual at age from 30 to 66 months,
the nutrient blend composition C comprising at least three ingredients selected from the group consisting of DHA, sphingomyelin, phosphatidylcholine, histidine, isoleucine, leucine, valine, alpha carotene, folic acid, alpha lactalbumin, gangliosides, phosphatidylinositol, phosphatidylethanolamine, eicosenoic acid, stearidonic acid, DPA, 3′-SL, 6′-SL, alanine, arginine, aspartic acid, cystine, glutamic acid, glycine, lysine, methionine, phenylalanine, proline, serine, threonine, tryptophan, tyrosine, alpha tocopherol, octadecatetraenoic acid, eicosapentaenoic acid, phosphorus, and phosphatidylserine.

14: The staged feeding regimen of claim 13, wherein the nutrient blend compositions A, B and C are administered in an amount effective to promote myelination of targeted brain regions.

15: The staged feeding regimen of claim 14, wherein the targeted brain region is at least one region of the social brain.
